(19) United States
(12) Patent Application Publication
(10) Pub. No.: US 2023/0265491 A1
(43) Pub. Date: Aug. 24, 2023

(54) SPATIAL TRANSCRIPTOMIC TRANSFER MODES

(71) Applicant: 10x Genomics, Inc., Pleasanton, CA (US)

(72) Inventors: Augusto Manuel Tentori, Dublin, CA (US); Hanyoup KIM, Foster City, CA (US); Rajiv Bharadwaj, Pleasanton, CA (US)

(21) Appl. No.: 17/923,007

(22) PCT Filed: Apr. 30, 2021
(86) PCT No.: PCT/US2021/030271

§ 371 Date: Nov. 3, 2022

Related U.S. Application Data
(60) Provisional application No. 63/019,719, filed on May 4, 2020.

(51) Int. Cl. C12Q 1/6837; C12Q 1/6869; C12Q 1/6841

(52) U.S. Cl. CPC C12Q 1/6837; C12Q 1/6869; C12Q 1/6841; C12Q 2600/16

(57) ABSTRACT

Provided herein are methods and systems for determining the location of one or more analytes in a biological sample with electrophoresis analyte transfer modes.

### CROSS-REFERENCE TO RELATED APPLICATIONS

This application claims priority to U.S. Provisional Patent Application No. 63/019,719, filed on May 4, 2020. The contents of this application is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Active capture methods, such as electrophoresis, facilitate spatially capturing analytes from a biological sample. Spatially capturing analytes via active capture methods can be accomplished in such a way to enable multi-omic assessment of analytes present in a biological sample.

### SUMMARY

Active capture methods, such as electrophoresis, facilitate spatially capturing analytes from a biological sample. Spatially capturing analytes via active capture methods can be accomplished in such a way to enable multi-omic assessment of analytes present in a biological sample. The present disclosure features methods for spatially capturing analytes in one or more semi-porous materials that can be further processed immediately or at a later time.

Thus, provided herein are methods for determining a location of one or more analytes in a biological sample, the method including: providing an array including a plurality of capture probes, where a capture probe of the plurality of capture probes includes (i) a spatial barcode and (ii) a capture domain; providing one or more semi-porous materials, where the one or more semi-porous materials are disposed between a biological sample and the array; applying an electric field to the biological sample, the one or more semi-porous materials, and the array, where the electric field promotes the migration of the one or more analytes in the direction of the array, where the capture domain of the capture probe specifically binds to a first analyte of the one or more analytes; and determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample.

In some embodiments, the capture probe further includes one or more of: a cleavage domain, a functional domain, and a unique molecular identifier. In some embodiments, the first analyte is mRNA.

In some embodiments, the method includes obtaining an image of the biological sample. In some embodiments, a first semi-porous material of the one or more semi-porous materials retains one or more second analytes. In some embodiments, the first semi-porous material includes a plurality of analyte-binding moieties, where an analyte-binding moiety of the plurality specifically binds to a second analyte of the one or more second analytes.

In some embodiments, the analyte binding moiety is an antibody or an antigen-binding fragment thereof. In some embodiments, the second analyte is a protein.

In some embodiments, the method includes determining the location of the second analyte in the first semi-porous material. In some embodiments, determining the location of the second analyte in the first semi-porous material includes the use of immunofluorescence staining. In some embodiments, determining the location of the second analyte in the first semi-porous material includes: obtaining an image of the first semi-porous material and correlating the immunofluorescence staining in the image of the first semi-porous material with an image of the biological sample.

In some embodiments, the one or more semi-porous materials includes a second semi-porous material, where the second semi-porous material retains one or more third analytes. In some embodiments, the second semi-porous material includes a plurality of analyte-binding moieties for the one or more third analytes. In some embodiments, the plurality of analyte binding moieties specifically binds to a third analyte of the one or more third analytes. In some embodiments, the third analyte is a nucleic acid. In some embodiments, the nucleic acid is miRNA. In some embodiments, the method includes determining the location of the third analyte in the second semi-porous material. In some embodiments, determining the location of the third analyte in the second semi-porous material includes the use of in situ hybridization. In some embodiments, determining the location of the third analyte includes: obtaining an image of the second semi-porous material and correlating the in situ hybridization in the image of the second semi-porous material with an image of the biological sample.

In some embodiments, the one or more semi-porous materials separate the one or more analytes from other molecules in the biological sample. In some embodiments, the one or more semi-porous materials include a hydrogel. In some embodiments, the one or more semi-porous materials includes a permeable membrane. In some embodiments, at least one of the one or more semi-porous materials has non-uniform pore sizes. In some embodiments, at least one of the one or more semi-porous materials has a substantially uniform pore size. In some embodiments, the electric field is applied to a discrete area of the biological sample. In some embodiments, the electric field is applied to a discrete area of the array.

In some embodiments, the one or more semi-porous materials includes one or more fiducial markers that align the one or more semi-porous materials to the image of the biological sample.

In some embodiments, the biological sample, the one or more semi-porous materials, and the array are in direct contact with a buffer. In some embodiments, the one or more second analytes to the first semi-porous material. In some embodiments, the method includes crosslinking the one or more third analytes to the second semi-porous material. In some embodiments, the method includes removing the first semi-porous material after the first semi-porous material retains the one or more second analytes. In some embodiments, the method includes removing the second semi-porous material after the second semi-porous material retains the one or more third analytes.

In some embodiments, the method includes: removing the first semi-porous material after the first semi-porous material retains the one or more second analytes; and disposing the second semi-porous material between the biological sample and the array.

In some embodiments, the electric field is applied after removal of the first semi-porous material and disposal of the second semi-porous material between the biological sample and the array.

In some embodiments, the second semi-porous material retains one or more third analytes.

In some embodiments, the biological sample is a tissue section. In some embodiments, the biological sample is a formalin-fixed paraffin-embedded biological sample. In some embodiments, the method includes a step of fixing the biological sample.

In some embodiments, the method includes a step of permeabilizing the biological sample.

Also provided herein are methods for determining a location of one or more analytes in a biological sample, the method including: (a) providing a first array including a plurality of first capture probes, where a first capture probe of the plurality of first capture probes includes (i) a first spatial barcode and (ii) a first capture domain; (b) providing a semi-porous material, where the semi-porous material is disposed between the biological sample and the first array; (c) applying an electric field to the biological sample, the semi-porous material, and the first array, where the electric field promotes the migration of the one or more analytes in the direction of the first array, where the first capture domain specifically binds to a first analyte of the one or more analytes, where the semi-porous material retains a second analyte of the one or more analytes; (d) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample; (e) providing a second array including a plurality of second capture probes, where a second capture probe of the plurality of second capture probes includes (i) a second spatial barcode and (ii) a second capture domain; (f) applying an electric field to the semi-porous material and the second array, where the electric field promotes the migration of the second analyte in the direction of the second array, where the second capture domain specifically binds to the second analyte; and (g) determining (i) all or a portion of the sequence of the second spatial barcode, or a complement thereof, and (ii) all or a portion of the sequence of the second analyte, or a complement thereof, and using the sequences of (i) and (ii) to determine the location of the second analyte in the biological sample.

In some embodiments, the capture probe further includes one or more of: a cleavage domain, a functional domain, and a unique molecular identifier. In some embodiments, the first analyte is mRNA.

In some embodiments, the method includes obtaining an image of the biological sample. In some embodiments, the semi-porous material includes one or more analyte binding moieties. In some embodiments, the one or more analyte binding moieties specifically bind to a nucleic acid. In some embodiments, where the nucleic acid is miRNA. In some embodiments, the semi-porous material retains one or more analytes from the biological sample. In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size.

In some embodiments, an electric field is applied to a discrete area of the biological sample. In some embodiments, the electric field is applied to a discrete area of the first array. In some embodiments, the electric field is applied to a discrete area of the second array. In some embodiments, the semi-porous material includes one or more fiducial markers that align the semi-porous material to the image of the biological sample.

In some embodiments, in step (c), the biological sample, the semi-porous material, and the first array are in direct contact with a buffer. In some embodiments, in step (f), the semi-porous material and the second array are in direct contact with a buffer.

In some embodiments, the method includes crosslinking the second analyte to the semi-porous material.

In some embodiments, the biological sample is a tissue section. In some embodiments, the biological sample is formalin-fixed and paraffin-embedded. In some embodiments, the method includes a step of fixing the biological sample. In some embodiments, the method includes a step of permeabilizing the biological sample.

Also provided herein are methods for determining a location of one or more analytes in a biological sample, the method including: (a) providing a first array including a plurality of first capture probes, where a first capture probe of the plurality of first capture probes includes (i) a first spatial barcode and (ii) a first capture domain; (b) providing a semi-porous material, where the semi-porous material is disposed between the biological sample and the first array, where the semi-porous material includes an analyte capture agent including (i) an analyte binding moiety, (ii) analyte binding moiety barcode, and (iii) an analyte capture sequence; (c) applying an electric field to the biological sample, the semi-porous material, and the first array, where the electric field promotes the migration of the one or more analytes in the direction of the first array, where the first capture domain specifically binds to a first analyte of the one or more analytes, and where the analyte binding moiety specifically binds to a second analyte of the one or more analytes, and the semi-porous material retains the second analyte; (d) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample; (e) providing a second array including a plurality of second capture probes, where a second capture probe of the plurality of second capture probes includes (i) a second spatial barcode and (ii) a second capture domain; (f) applying an electric field to the semi-porous material and the second array, where the electric field promotes the migration of the second analyte in the direction of the second array, where the second capture domain specifically binds to the analyte capture sequence; (g) determining (i) all or a portion of the sequence of the second spatial barcode, or a complement thereof, and (ii) all or a portion of the sequence of the analyte binding moiety barcode, or a complement thereof, and using the sequences of (i) and (ii) to determine the location of the second analyte in the biological sample.

In some embodiments, one or both of the first capture probe and the second capture probe further includes one or more of a cleavage domain, a functional domain, and a unique molecular identifier. In some embodiments, the first analyte is mRNA.

In some embodiments, the method includes obtaining an image of the biological sample.

In some embodiments, the analyte capture agent is crosslinked to the semi-porous material. In some embodiments, the second analyte is a protein.

In some embodiments, the analyte binding moiety includes an antibody or an antigen-binding fragment thereof.

In some embodiments, the semi-porous material separates one or more analytes from other molecules in the biological sample. In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size. In some embodiments, the electric field is applied to a discrete area of the biological sample. In some embodiments, the electric field is applied to a discrete area of the first array. In some embodiments, the electric field is applied to a discrete area of the second array.

In some embodiments, the semi-porous material includes one or more fiducial markers that align the semi-porous material to the image of the biological sample.

In some embodiments, during step (c), the biological sample, the semi-porous material, and the first array, are in direct contact with a buffer. In some embodiments, during step (f), the semi-porous material and the second array are in direct contact with a buffer.

In some embodiments, the method includes crosslinking the second analyte to one or both of the semi-porous material and the analyte capture agent.

In some embodiments, the biological sample is a tissue section. In some embodiments, the biological sample is formalin-fixed and paraffin-embedded. In some embodiments, the method includes a step of fixing the biological sample. In some embodiments, the method includes a step of permeabilizing the biological sample.

Also, provided herein are systems including: (a) an array including a plurality of capture probes, where a capture probe of the plurality of capture probes includes (i) a spatial barcode and (ii) a capture domain; (b) one or more semi-porous materials disposed between a biological sample and the array; and (c) an electric field, where the electric field promotes the migration of one or more analytes in the direction of the array, where the capture domain of the capture probe binds to a first analyte of the one or more analytes.

In some embodiments, a first semi-porous material of the one or more semi-porous materials retains one or more second analytes. In some embodiments, the first semi-porous material includes a plurality of analyte-binding moieties, where an analyte-binding moiety of the plurality specifically binds to a second analyte of the one or more second analytes. In some embodiments, the analyte binding moiety is an antibody or antigen-binding fragment thereof. In some embodiments, the second analyte is a protein.

In some embodiments, the method includes determining the location of the second analyte in the first semi-porous material. In some embodiments, the method includes determining the location of the second analyte in the first semi-porous material includes immunofluorescence staining.

In some embodiments, determining the location of the second analyte in the first semi-porous material includes: obtaining an image of the first semi-porous material; and correlating the immunofluorescence staining in the image of the first semi-porous material with an image of the biological sample.

In some embodiments, the one or more semi-porous materials includes a second semi-porous material, where the second semi-porous material retains one or more third analytes. In some embodiments, the second semi-porous material includes a plurality of analyte-binding moieties for the one or more third analytes. In some embodiments, an analyte-binding moiety of the plurality of analyte binding moieties specifically binds to a third analyte of the one or more third analytes. In some embodiments, the third analyte is a nucleic acid. In some embodiments, the nucleic acid is miRNA.

In some embodiments, the method includes determining the location of the third analyte in the second semi-porous material. In some embodiments, determining the location of the third analyte in the second semi-porous material includes the use of in situ hybridization. In some embodiments, determining the location of the third analyte includes: obtaining an image of the second semi-porous material and correlating the in situ hybridization in the image of the second semi-porous material with an image of the biological sample.

In some embodiments, the method includes crosslinking the one or more third analytes to the second semi-porous material. In some embodiments, the method includes cross-linking the one or more second analytes to the first semi-porous material. In some embodiments, the method includes removing the second semi-porous material after the second semi-porous material retains the one or more third analytes.

In some embodiments, the method includes removing the first semi-porous material after the first semi-porous material retains the one or more second analytes; and disposing the second semi-porous material between the biological sample and the array.

In some embodiments, the electric field is applied after removal of the first semi-porous material and disposal of the second semi-porous material between the biological sample and the array.

In some embodiments, the second semi-porous material retains one or more third analytes.

In some embodiments, the capture probe further includes one or more of: a cleavage domain, a functional domain, and a unique molecular identifier.

In some embodiments, the method includes imaging the biological sample.

In some embodiments, the first analyte is mRNA

In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size.

In some embodiments, the one or more semi-porous materials include one or more fiducial markers that align the one or more semi-porous materials to the image of the biological sample.

In some embodiments, the biological sample is a tissue section. In some embodiments, the biological sample is a formalin-fixed paraffin-embedded biological sample. In some embodiments, the method includes a step of fixing the biological sample. In some embodiments, the method includes a step of permeabilizing the biological sample.

In some embodiments, the second analyte is protein.

In some embodiments, an electric field is applied to a discrete area of the biological sample. In some embodiments, the electric field is applied to a discrete area of the array. In some embodiments, the biological sample, the semi-porous material, and the array, are in direct contact with a buffer. In some embodiments, the system further includes determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample.

Also provided herein are system including: (a) a first array including a plurality of first capture probes, where a first capture probe of the plurality of first capture probes includes (i) a first spatial barcode and (ii) a first capture domain; (b) a semi-porous material, where the semi-porous material is disposed between the biological sample and the first array; (c) a first electric field, where the first electric field promotes the migration of the one or more analytes in the direction of the first array, where the first capture domain specifically binds to a first analyte of the one or more analytes, where the semi-porous material retains a second analyte of the one or more analytes; (d) a second array including a plurality of second capture probes, where a second capture probe of the plurality of second capture probes includes (i) a second spatial barcode and (ii) a second capture domain; (e) a second electric field, where the second electric field promotes the migration of the second analyte in the direction of the second array, where the second capture domain specifically binds to the second analyte; and (f) determining (i) all or a portion of the sequence of the second spatial barcode, or a complement thereof, and (ii) all or a portion of the sequence of the second analyte, or a complement thereof, and using the sequences of (i) and (ii) to determine the location of the second analyte in the biological sample.

In some embodiments, the capture probe further includes one or more of: a cleavage domain, a functional domain, and a unique molecular identifier. In some embodiments, the first analyte is mRNA.

In some embodiments, the system further includes obtaining an image of the biological sample.

In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size.

In some embodiments, the one or more semi-porous materials include one or more fiducial markers that align the one or more semi-porous materials to the image of the biological sample.

In some embodiments, the biological sample is a tissue section. In some embodiments, the biological sample is a formalin-fixed paraffin-embedded biological sample. In some embodiments, the method includes a step of fixing the biological sample. In some embodiments, the method includes a step of permeabilizing the biological sample.

In some embodiments, the semi-porous material retains one or more analytes from the biological sample.

In some embodiments, the first electric field, the second electric field, or both, is applied to a discrete area of the biological sample. In some embodiments, the first electric field is applied to a discrete area of the first array. In some embodiments, the second electric field is applied to a discrete area of the second array.

In some embodiments, the biological sample, the semi-porous material, and the first array are in direct contact with a buffer. In some embodiments, the biological sample, the semi-porous material and the second array are in direct contact with a buffer. In some embodiments, the semi-porous material separates one or more analytes from other molecules in the biological sample. In some embodiments, the semi-porous material includes one or more analyte binding moieties.

In some embodiments, the one or more analyte binding moieties bind a nucleic acid. In some embodiments, the nucleic acid is miRNA. In some embodiments, the analyte binding moiety is crosslinked to the semi-porous material.

In some embodiments, the semi-porous material includes one or more analyte capture agents. In some embodiments, the one or more analyte capture agents includes (i) an analyte binding moiety, (ii) analyte binding moiety barcode, and (iii) an analyte capture sequence. In some embodiments, the second analyte is protein.

In some embodiments, the analyte capture agent is crosslinked to the semi-porous material.

In some embodiments, the analyte binding moiety include an antibody or an antigen-binding fragment thereof.

In some embodiments, the analyte binding moiety barcode identifies the analyte capture agent. In some embodiments, the analyte capture sequence binds to the second capture domain of the second capture probe.

In some embodiments, the system further includes: (a) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample; and (b) determining (i) all or a portion of the sequence of the second spatial barcode, or a complement thereof, and (ii) all or a portion of the sequence of the analyte binding moiety barcode, or a complement thereof, and using the sequences of (i) and (ii) to determine the location of the second analyte in the biological sample.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram of an exemplary analyte capture agent.

FIG. 3 is a schematic diagram depicting an exemplary interaction between a feature-immobilized capture probe 324 and an analyte capture agent 326.

FIG. 4A shows an exemplary configuration for migrating analytes using the methods described herein.

FIG. 4B shows a diagram showing the migration of analytes using an example of the methods described herein.

FIG. 4C shows the results of migrating analytes using the exemplary methods depicted in FIG. 4B.

FIG. 5 shows an example schematic workflow for spatially transferring analytes where a semi-porous material is used as a filter.

FIG. 6 shows an example schematic workflow for spatially transferring analytes where a semi-porous material is used as a molecular sieve.

FIG. 7A shows an example schematic workflow for spatially transferring analytes where a semi-porous material has discontinuities in pore size.

FIG. 7B shows an example schematic workflow for spatially transferring analytes where a semi-porous material has gradients in pore size.

FIG. 8 shows an example schematic workflow for spatially transferring analytes where discontinuous buffers are used for isotachophoresis (ITP).

FIG. 9 shows an example schematic workflow for spatially transferring analytes where the buffer only contacts a first region of the biological sample so that only analytes from the first region are transferred to the capture probes.

### DETAILED DESCRIPTION

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample such as a mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

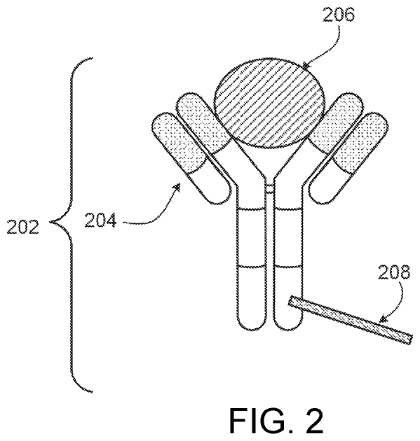

FIG. 2 is a schematic diagram of an exemplary analyte capture agent 202 comprised of an analyte binding moiety 204 and a capture agent barcode domain 208. An analyte binding moiety 204 is a molecule capable of binding to an analyte 206 and interacting with a spatially-barcoded capture probe. The analyte binding moiety can bind to the analyte 206 with high affinity and/or with high specificity. The analyte capture agent can include a capture agent barcode domain 208, a nucleotide sequence (e.g., an oligonucleotide), which can hybridize to at least a portion or an entirety of a capture domain of a capture probe. The analyte binding moiety 204 can include a polypeptide and/or an aptamer (e.g., an oligonucleotide or peptide molecule that binds to a specific target analyte). The analyte binding moiety 204 can include an antibody or antibody fragment (e.g., an antigen-binding fragment).

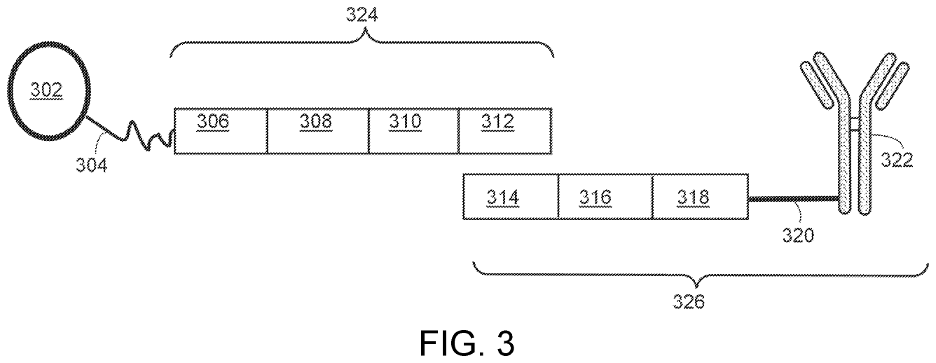

FIG. 3 is a schematic diagram depicting an exemplary interaction between a feature-immobilized capture probe 324 and an analyte capture agent 326. The feature-immobilized capture probe 324 can include a spatial barcode 308 as well as one or more functional sequences 306 and 310, as described elsewhere herein. The capture probe can also include a capture domain 312 that is capable of binding to an analyte capture agent 326. The analyte capture agent 326 can include a functional sequence 318, capture agent barcode domain 316, and an analyte capture sequence 314 that is capable of binding to the capture domain 312 of the capture probe 324. The analyte capture agent can also include a linker 320 that allows the capture agent barcode domain 316 to couple to the analyte binding moiety 322.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

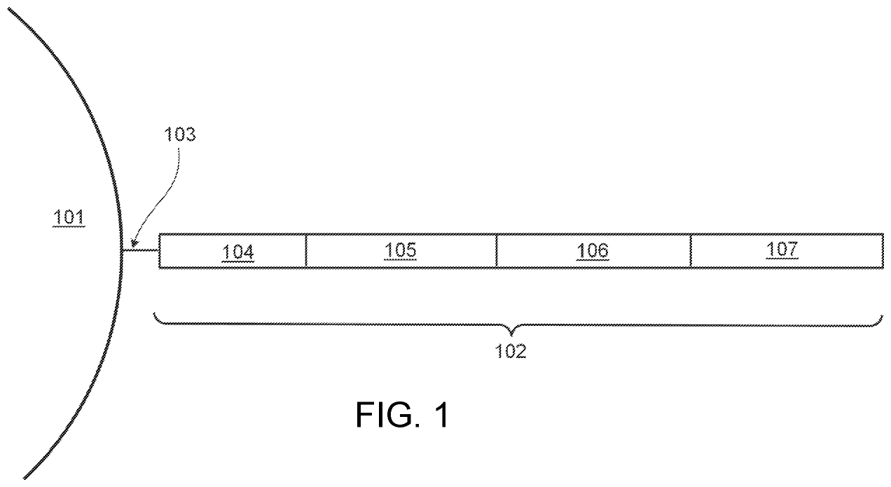

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Spatially Transferring Analytes and Multi-Omic Spatial Analysis

Provided herein are methods for spatially determining the location of analytes (e.g., any of the exemplary analytes described herein) in a biological sample (e.g., any of the exemplary biological samples described herein), the method including providing an array (e.g., any of the arrays described herein) having a plurality of capture probes, where a first capture probe of the plurality of capture probes has (i) a spatial barcode and (ii) a capture domain (e.g., any of the exemplary capture domains described herein), providing one or more semi-porous material(s) (e.g., any of the exemplary semi-porous materials described herein) where the one or more semi-porous materials are disposed between the biological sample and the array, applying an electric field to the biological sample, the one or more porous materials, and the array, where the electric field promotes the migration of the one or more analytes in the direction of the array, where the capture domain of the capture probe specifically binds to a first analyte of the one or more analytes; and determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample.

In some embodiments, when the one or more analytes migrate in the direction of the array, the one or more semi-porous materials between the biological sample and the array can retain one or more second analytes. As used herein, the word “retain” indicates that the retained analyte either migrates slower in the semi-porous material (e.g., as compared to another analyte or as compared to the same analyte before it enters the semi-porous material) or is prevented from migrating through the semi-porous material after entering the semi-porous material. In some embodiments, the one or more semi-porous materials between the biological sample and the array can retain one or more second analytes non-specifically based on physicochemical properties such as, for example, charge, size (e.g., length, radius of gyration, effective diameters, etc.), hydrophobicity/hydrophilicity, molecular binding (e.g., immunoaffinity), or any combination thereof. For example, the one or more semi-porous materials between the biological sample and the array can contain a plurality of analyte capture agents (e.g., any of the exemplary analyte capture agents described herein). In some embodiments, the one or more semi-porous materials between the biological sample and the array contain a plurality of analyte capture agents that includes an analyte-binding moiety (e.g., any of the exemplary analyte-binding moieties described herein) where each analyte-binding moiety can specifically bind a second analyte of the one or more second analytes.

In some embodiments, the biological sample is a tissue section. In some embodiments, the biological sample is a formalin-fixed and paraffin-embedded biological sample. In some embodiments, the methods described herein can further include fixing the biological sample. In some embodiments, the methods described herein can further include permeabilizing the biological sample. In some embodiments, the one or more analytes (e.g., first analytes and/or second analytes) can be RNA, DNA, protein, miRNA, siRNA, rRNA, mtRNA, snoRNAs, and any other type of analyte described herein. In some embodiments, the capture probe can further include one or more of: a cleavage domain, one or more functional domains, a unique molecular identifier, or combinations thereof.

In some embodiments, the methods described herein can further include obtaining an image of the biological sample. In some embodiments, the biological sample is fixed by a fixation buffer. In some embodiments, the biological sample is permeabilized by a permeabilization buffer. In some embodiments, the biological sample is stained by H&E (haematoxylin and eosin) before it is imaged. In some embodiments, the methods described herein can further include obtaining an image of the biological sample with a microscope. In some embodiments, the one or more semi-porous materials contain one or more fiducial markers that align the one or more semi-porous materials to the image of the biological sample. In some embodiments, one or more analytes can be imaged during migration through the one or more semi-porous materials.

Some embodiments of the methods described herein can include active migration of one or more analytes. For example, an electric field can be applied to the array, the biological sample, or both to promote the migration of analytes. In some embodiments, an electric field can be applied to the array, the biological sample, and one or more semi-porous materials. In some embodiments, the analytes are migrated into one or more semi-porous materials. In some embodiments, the electric field can be applied to a discrete area of the biological sample (e.g., a region of interest in the biological sample). Some embodiments of the methods described herein can further include identifying a region of interest in the biological sample. In some embodiments, an electric field can be applied to a discrete area of the array.

In some embodiments, after the one or more semi-porous materials between the biological sample and the array retain one or more second analytes, the one or more semi-porous materials containing the retained second analytes can be removed for further analysis. In some embodiments, methods described herein can further include providing one or more new semi-porous materials after the one or more semi-porous materials containing the retained second analytes are removed and applying a new electric field, so that the new electric field promotes the migration of one or more third analytes in the direction of the array. In some embodiments, the one or more new semi-porous materials can retain the one or more third analytes.

a) Substrate Holder

In some embodiments, the biological sample is placed on or in a substrate holder (e.g., a cassette). For example, the biological sample can be placed on a substrate (e.g., any of the substrates described herein), which can then be placed on or in a substrate holder (e.g., a cassette).

A substrate holder, such as a cassette, can be used to ensure that the temperature of a substrate and any biological samples and/or reagents supported on the substrate surface is controlled uniformly and consistently during a sample preparation and/or analysis protocol. During such protocols, uneven heating can lead to failure of the sample preparation. Further, even when sample heating is relatively uniform, condensation that contacts the sample may impair certain reactions that are part of the protocol, or otherwise affect the chemical reactions that occur. The substrate holder, in various embodiments, provide for heating and/or generating an electric field of multiple surfaces of a substrate (e.g., in a slide cassette or substrate holder), and can include features that facilitate heat transfer from heating elements to the substrate, and that can reduce or prevent condensation from forming in certain regions of the substrate (e.g., in sample wells or regions on the substrate surface). In some embodiments, the substrate holder (e.g., a cassette) is used to generate an electric field (e.g., a first electric field, a second electric field) to promote the migration of analytes from the biological sample to an array (e.g., a first array, a second array). In some embodiments, one or more semi-porous materials is disposed between the biological sample and the array (e.g., a first array, a second array) and the biological sample, the one or more semi-porous materials, and the array can be heated and/or an electric field can be generated to promote the migration of one or more analytes from the biological sample to the array.

In some embodiments, the biological sample is placed on a conductive substrate. In some embodiments, the biological sample is placed on a semi-porous substrate. In some embodiments, the biological sample is placed on a semi-porous membrane. In some embodiments, the biological sample is placed on a nitrocellulose membrane. In some embodiments, the biological sample is placed on a slide. In some embodiments, the biological sample is placed on a pathology glass slide. In some embodiments, the biological sample is placed on a conductive plate. In some embodiments, the biological sample is placed on an indium tin oxide (ITO) coated slide. In some embodiments, the biological sample is placed on an ITO coated poly-Lysine slide.

In some embodiments, the array is placed on a substrate holder. For example, the array having a plurality of capture probes includes capture probes on a substrate (e.g., any of the substrates described herein), which can then be placed on the substrate holder. In some embodiments, the array having a plurality of capture probes includes capture probes on features (e.g., any feature described herein) on a substrate. In some embodiments, the array is on a conductive substrate. In some embodiments, the array having a plurality of capture probes is placed on a semi-porous substrate. In some embodiments, the array having a plurality of capture probes is placed on a semi-porous membrane. In some embodiments, the array having a plurality of capture probes is placed on a nitrocellulose membrane. In some embodiments, the array having a plurality of capture probes is placed on a slide. In some embodiments, the array having a plurality of capture probes is placed on a pathology glass slide. In some embodiments, the array having a plurality of capture probes is placed on a conductive plate. In some embodiments, the array having a plurality of capture probes is placed on an ITO coated slide. In some embodiments, the array having a plurality of capture probes is placed in discrete areas of the substrate.

In some embodiments, there is a gap (e.g., a space) between the array and the biological sample. In some embodiments, the gap between the array and the biological sample is filled with one or more solutions. In some embodiments, the one or more solutions between the substrate containing the array and the biological sample can include a permeabilization buffer (e.g., any of the permeabilization buffers described herein). In some embodiments, the one or more solutions between the array and the biological sample can include an electrophoresis buffer. For example, an electrophoresis buffer is a buffer that can facilitate a current and maintain the pH at a relatively constant value.

In some embodiments, the biological sample, the one or more semi-porous materials, and the array are all in direct contact with a buffer (e.g., permeabilization buffer, electrophoresis buffer). In some embodiments, the biological sample, the one or more semi-porous materials, and the array are all in direct contact with a permeabilization buffer. In some embodiments, the biological sample is in direct contact with an electrode. In some embodiments, the array is in direct contact with an electrode. In some embodiments the biological sample is separated from an electrode by a buffer (e.g., permeabilization buffer, electrophoresis buffer). In some embodiments, the array is separated from an electrode by a buffer (e.g., permeabilization buffer, electrophoresis buffer).

In some embodiments, a biological sample can be placed in a first substrate holder (e.g., a substrate holder described herein). In some embodiments, a spatially-barcoded capture probe array (e.g., capture probes, barcoded array) can be placed on a second substrate holder (e.g., a substrate holder described herein). In some embodiments, a biological sample can be placed in a first substrate holder that also contains capture probes. In some embodiments, the first substrate holder, the second substrate holder, or both can be conductive (e.g. any of the conductive substrates described herein). In some embodiments, the first substrate holder including the biological sample, the second substrate holder including an array having capture probes, or both, can be contacted with permeabilization reagents (e.g., a permeabilization buffer) and/or an electrophoresis buffer and analytes can be migrated from the biological sample toward the array using an electric field.

In some embodiments, electrophoresis can be applied to a biological sample on an array while in contact with a permeabilization buffer. In some embodiments, electrophoresis can be applied to a biological sample on an array while in contact with an electrophoresis buffer. In some embodiments, electrophoresis can be applied to a biological sample on an array while in contact with an electrophoresis buffer (e.g. a buffer that lacks permeabilization reagents). In some embodiments, the permeabilization buffer can be replaced with an electrophoresis buffer after a desired amount of time. In some embodiments, electrophoresis can be applied simultaneously with the permeabilization buffer and/or electrophoresis buffer. In some embodiments, electrophoresis can be applied after a desired amount of time of contact between the biological sample and the permeabilization buffer and/or electrophoresis buffer.

In some embodiments, the first substrate holder and the second substrate holder can facilitate alignment of the biological sample, the one or more semi-porous materials, and the array with each other. In some embodiments, the biological sample is in direct contact with the one or more semi-porous materials. In some embodiments, the array is in direct contact with the one or more semi-porous materials. In some embodiments, both the biological sample and the array are in direct contact with the one or more semi-porous materials.

b) Semi-Porous Material

In some embodiments, the methods described herein can include transferring analytes into a semi-porous material between the biological sample and the array. In some embodiments, the methods described herein can include the use of two, three, four, or more semi-porous materials (e.g., the same of different semi-porous materials) between the biological sample and the array.

In some embodiments, the methods described herein can include one semi-porous material between the biological sample and the array. In some embodiments, the semi-porous material can be a permeable membrane. In some embodiments, the semi-porous material can be a hydrogel or an organogel. For example, a semi-porous material can be a SDS-PAGE gel. In some embodiments, the semi-porous material can have a substantially uniform pore size. In some embodiments, the semi-porous material can have non-uniform pore sizes. For example, a semi-porous material can be a gradient gel. In some embodiments, the semi-porous material can contain one or more permeabilization reagents (e.g., one or more of any of the exemplary permeabilization reagents described herein) (e.g., permeabilization buffer, dried permeabilization reagents). In some embodiments, the semi-porous material can contain an electrophoresis buffer. In some embodiments, one or more first analytes (e.g., nucleic acid, DNA, RNA, or any of the other exemplary analytes described herein) from the biological sample can be transferred through the semi-porous material (e.g., one or more first analytes that are not retained in the semi-porous material) with an electric field and specifically bind (e.g., hybridize) capture probes on the array.

In some embodiments, the semi-porous material can separate one or more analytes from other molecules from the biological sample. Here, the term “molecules” refers to any molecules from a biological sample. In some embodiments, in a non-limiting way, the molecules from the biological sample include proteins, nucleic acids, lipids, carbohydrates, and small molecules such as water, adenosine triphosphate, salts, organic acids, metal ions, etc. For example, the semi-porous material can separate nucleic acids from proteins based on molecular size. In some embodiments, the semi-porous material can separate shorter nucleic acids (e.g., miRNAs) from longer nucleic acids (e.g., mRNAs).

In some embodiments, the semi-porous material(s) between the biological sample and the array can act as a filter to separate analytes (e.g., analytes of interest) from other molecules or analytes present in the biological sample. In some embodiments, the analytes (e.g., analytes of interest) are RNA transcripts (e.g., mRNA). In some embodiments, the semi-porous material(s) between the biological sample and the array can act as a filter to separate mRNA from other molecules (e.g., analytes) such as proteins, lipids and/or other nucleic acids. In some embodiments, the semi-porous material(s) between the biological sample and the array can act as a filter to separate the analytes and other molecules based on physicochemical properties. In some embodiments, analytes can be separated based on properties such as charge, size (e.g., length, radius of gyration, effective diameter, etc.), hydrophobicity, hydrophilicity, molecular binding (e.g., immunoaffinity), or combinations thereof. In some embodiments, the semi-porous material(s) between the biological sample and the array can separate the analytes from other molecules to reduce non-specific binding near the capture probes and therefore improve binding between the analytes and the capture probes of the array, thus improving subsequent assay performance.

In some embodiments, the semi-porous material(s) between the biological sample and the array can act as a molecular sieve matrix for electrophoretic analyte separation. For example, separation of analytes can occur based on physicochemical properties such as charge, size (e.g., length, radius of gyration, and effective diameters, etc.), electrophoretic mobility, zeta potential, isoelectric point, hydrophobicity, hydrophilicity, molecular binding (e.g., immunoaffinity), and combinations thereof. In some embodiments, the semi-porous material(s) between the biological sample and the array can have a uniform pore size. In some embodiments, the semi-porous material(s) between the biological sample and the array can have discontinuities in pore size, as generally used in different gel electrophoresis schemes. In some embodiments, the semi-porous material(s) between the biological sample and the array can have gradients in pore sizes. For example, the semi-porous material(s) (e.g., a hydrogel) can have a gradient of pore sizes such that the gradient separates the analytes as the analytes migrate to the array.

In some embodiments, different molecules, including analytes, in the biological sample move through the semi-porous material(s) at different rates. For example, a smaller molecule can move through the semi-porous material faster relative to a larger molecule. In some embodiments, after applying the electric field for a certain amount of time, only one or more analytes or types of analytes (e.g., mRNA) can move through the one semi-porous material(s) to reach the array while other molecules are retained in the semi-porous material(s). In some embodiments, by controlling the amount of time the electric field is applied, the one or more analytes can be selectively migrated through the semi-porous material(s) and specifically bind (e.g., hybridize) capture probes of the array. In some embodiments, the migration (e.g., migration rate) of the molecules in the biological sample can be controlled by varying the intensity of the electric field applied to the biological sample, or varying the semi-porous material(s). In some embodiments, controlling the intensity of the electric field and the time the electric field is applied, one or more analytes can be selectively migrated through the semi-porous material. In some embodiments, the electric field is removed after the one or more analytes (e.g., mRNA) have migrated through the semi-porous material. In some embodiments, the semi-porous material is removed after the one or more analytes (e.g., mRNA) have migrated through the semi-porous material.

In some embodiments, the semi-porous material(s) can retain one or more second analytes. In some embodiments, the semi-porous material(s) can retain one or more second analytes through specific binding. For example, the semi-porous material(s) can contain a plurality of analyte capture agents (e.g., any of the exemplary analyte capture agents described herein) that include an analyte-binding moiety (e.g., any of the exemplary analyte-binding moieties described herein) where each analyte-binding moiety can specifically bind a second analyte of the one or more second analytes. In some embodiments, the analyte-binding moieties can be antibodies or antigen-binding fragments (e.g., a Fab) thereof. In some embodiments, the one or more second analytes can be proteins (e.g., any of the exemplary proteins described herein). In some embodiments, the analyte-binding moieties can specifically bind certain nucleic acids or types of nucleic acids. In some embodiments, the one or more second analytes are nucleic acids. In some embodiments, the semi-porous material is removed after it retains the one or more second analytes. In some embodiments, the electric field is removed after the semi-porous material retains the one or more second analytes. In some embodiments, the analyte-binding moieties are cross-linked to the semi-porous material. In some embodiments, the analyte-binding moieties are cross-linked to the semi-porous material via photoreactive cross-linkers. Non-limiting examples of photoreactive cross-linkers include aryl azides (phenyl azides) and diazirines. Additional examples of photoreactive cross-linkers are known in the art.

In some embodiments, the semi-porous material(s) can retain one or more second analytes non-specifically. In some embodiments, the semi-porous material(s) can retain one or more second analytes based on physicochemical properties such as charge, size (length, radius of gyration, effective diameters, etc.), hydrophobicity/hydrophilicity, molecular binding (e.g., immunoaffinity), or any combination thereof. In some embodiments, the semi-porous material(s) can retain all molecules that are larger than a threshold molecular size (e.g., 1 kDa, 2 kDa, 3 kDa, 4 kDa, 5 kDa, 6 kDa, 8 kDa, 10 kDa, 12 kDa, 14 kDa, 16 kDa, 18 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, or 100 kDa). In some embodiments, the semi-porous material(s) can retain all nucleic acids that are longer than a threshold number of nucleotides (e.g., 25 nucleotides, 50 nucleotides, 100 nucleotides, 150 nucleotides, 200 nucleotides, 250 nucleotides, 300 nucleotides, 350 nucleotides, 400 nucleotides, 500 nucleotides, 750 nucleotides, 1,000 nucleotides, 2,000 nucleotides, 4,000 nucleotides, 6,000 nucleotides, 8,000 nucleotides, 10,000 nucleotides, 15,000 nucleotides, 20,000 nucleotides, 25,000 nucleotides, 30,000 nucleotides, 35,000 nucleotides, 40,000 nucleotides, 45,000 nucleotides, or 50,000 nucleotides). In some embodiments, different molecules in the biological sample migrate through the semi-porous material(s) at different rates. In some embodiments, after applying the electric field for a certain period of time (e.g., about 5 minutes to about 10 hours), one or more second analytes are retained in the semi-porous material while other analytes can have moved through the semi-porous material and/or have yet to enter the semi-porous material. In some embodiments, by controlling the amount of time the electric field is applied, the one or more second analytes can be selectively retained in the semi-porous material(s). In some embodiments, the migration (e.g., migration rate) of the molecules in the biological sample and/or the semi-porous material(s) can be controlled by varying the intensity of the electric field applied to the biological sample, the semi-porous material, and the array. In some embodiments, by controlling the intensity of the electric field and the time the electric field is applied, the one or more second analytes can be selectively retained in the semi-porous material(s). In some embodiments, the electric field is removed after the semi-porous material(s) retains the one or more second analytes of interest. In some embodiments, the semi-porous material(s) can be removed after the semi-porous material(s) retain the one or more second analytes of interest. In some embodiments, the semi-porous material(s) can retain one or more second analytes both non-specifically and through specific binding. In some embodiments, the semi-porous materials can include analyte-binding moieties that are cross-linked to the semi-porous material(s). In some embodiments, the analyte-binding moieties are cross-linked to the one semi-porous material via photoreactive cross-linkers (e.g., any of the exemplary photoreactive cross-linkers described herein).

c) Determining the Location of the One or More Analytes

In some embodiments, one or more first analytes migrate through the semi-porous material(s) and specifically bind (e.g., hybridize) to the capture domains on the capture probes of the array. In some embodiments, one or more mRNAs migrate through the semi-porous material(s) and bind (e.g., hybridize) to the capture domains on the capture probes on the array. In some embodiments, the sequence of all or a portion of the capture probes (e.g., the spatial barcode or a portion thereof) or a complement thereof, on the array and all or a portion of the sequence of the corresponding captured first analyte (or a complement thereof) are determined. In some embodiments, the location of the one or more first analytes in the biological sample are determined based on all or a portion of the sequence of the capture probes (e.g., the spatial barcode or a portion thereof) on the array, or a complement thereof, and all or a portion of the sequence of the corresponding captured one or more first analytes, or a complement thereof. In some embodiments, determining the location of the one or more first analytes in the biological sample includes determining all or a portion of the sequence of the capture probes (e.g., the spatial barcode, or a portion thereof) on the array, or a complement thereof, and all or a portion of the sequence of the corresponding captured one or more first analytes, or complement thereof, and correlating such sequence information to an image of the biological sample. Some embodiments of these methods further include obtaining an image of the biological sample.

In some embodiments, the semi-porous material(s) between the biological sample and the array retains one or more second analytes. In some embodiments, the location of the one or more second analytes retained in the semi-porous material(s) are determined by immunofluorescence. In some embodiments, one or more detectable labels (e.g., fluorophore-labeled antibodies) bind to the one or more second analytes that are retained in the semi-porous material and the location of the one or more second analytes is determined by detecting the labels under suitable conditions. In some embodiments, one or more fluorophore-labeled antibodies bind to the one or more second analytes that are retained in the semi-porous material(s) and the location(s) of the one or more second analytes is determined by imaging the fluorophore-labeled antibodies when the fluorophores are excited by a light of a suitable wavelength. In some embodiments, the location of the one or more second analytes in the biological sample is determined by correlating the immunofluorescence data to an image of the biological sample. In some embodiments, one or more nucleic acids (e.g., miRNA) are retained in the semi-porous material(s). In some embodiments, the location of the one or more second analytes that are retained in the semi-porous material(s) is determined by in situ hybridization (e.g., in situ hybridization using a labelled probe). In some embodiments, the location of the one or more second analytes in the biological sample is determined by correlating an image of the in situ hybridization to an image of the biological sample.

In some embodiments, the methods described herein can further include providing a second array that contains a plurality of second capture probes. In some embodiments, a second capture probe of the plurality of second capture probes contains (i) a second spatial barcode and (ii) a second capture domain that can bind (e.g., hybridize) to a second analyte. In some embodiments, the methods described herein can further include transferring analytes by applying a second electric field (e.g., an electric field applied after a first electric field) to the semi-porous material containing the one or more second analytes and the second array so that the electric field promotes the migration of the one or more second analytes in the direction of the second array. In some embodiments, the one or more second analytes bind (e.g., hybridize) to the second capture domains on the second capture probes on the second array. In some embodiments, the one or more second analytes can be miRNAs or any of the other types of analytes described herein. In some embodiments, all or a portion of the sequence of the second capture probes (e.g., all or a portion of the second spatial barcode), on the second array, or a complement thereof, and all or a portion of the sequence of the corresponding captured second analytes, or a complement thereof, are determined. In some embodiments, the location of the one or more second analytes in the biological sample are determined based on all or a portion of the sequence of the second capture probes (e.g., all or a portion of the spatial barcode) of the second capture probes on the second array, or a complement thereof, and all or a portion of the sequence of the corresponding captured one or more second analytes, or a complement thereof. In some embodiments, determining the location of the one or more second analytes in the biological sample includes determining all or a portion of the sequence of the second capture probes (e.g., all or a portion of the spatial barcode) on the second array, or complement thereof, and all or a portion of the sequence of the corresponding captured one or more second analytes, or a complement thereof, and correlating this sequence information to an image of the biological sample.

d) Two or More Semi-Porous Materials

In some embodiments, the methods described herein can include the use of two or more semi-porous materials between the biological sample and the array. In some embodiments, a first semi-porous material is disposed between the biological sample and a second semi-porous material. In some embodiments, the second semi-porous material is disposed between the first semi-porous material and the array. In some embodiments, at least one of the two semi-porous materials can separate one or more analytes from other molecules in the biological sample. In some embodiments, the one or more analytes from the biological sample migrate in the following direction: the biological sample, the first semi-porous material, the second semi-porous material, and the array. In some embodiments, one or more first analytes from the biological sample can move through the two semi-porous materials and reach the array.

In some embodiments, at least one of the two semi-porous materials can be a permeable membrane. In some embodiments, at least one of the two semi-porous materials can be a gel (e.g., a hydrogel or an organogel). For example, at least one of the two semi-porous materials can be a SDS-PAGE gel. In some embodiments, at least one of the two semi-porous materials can have a substantially uniform pore size. In some embodiments, at least one of the two semi-porous materials can have non-uniform pore sizes. For example, at least one of the two semi-porous materials can be a gradient gel. In some embodiments, at least one of the two semi-porous materials can contain a permeabilization buffer. In some embodiments, at least one of the two semi-porous materials can contain an electrophoresis buffer.

In some embodiments, at least one of the two semi-porous materials can separate one or more analytes from other molecules from the biological sample. For example, at least one of the two semi-porous materials can separate nucleic acids from proteins based on molecular size.

In some embodiments, at least one of the two semi-porous materials between the biological sample and the array can act as a filter to separate analytes (e.g., analytes of interest) from other molecules or analytes present in the biological sample. In some embodiments, the analytes (e.g., analytes of interest) are RNA (e.g., mRNA). In some embodiments, at least one of the two semi-porous materials between the biological sample and the array can act as a filter to separate RNA (e.g., mRNA) from other molecules (e.g., analytes) such as proteins, lipids, and/or other nucleic acids. In some embodiments, at least one of the two semi-porous materials between the biological sample and the array can act as a filter to separate the analytes and other molecules based on physicochemical properties. For example, analytes can be separated based on properties such as charge, size (e.g., length, radius of gyration, effective diameters, etc.), hydrophobicity, hydrophilicity, molecular binding (e.g., immunoaffinity), or any combination thereof. In some embodiments, at least one of the two semi-porous materials between the biological sample and the array can separate the analytes from other molecules to reduce non-specific binding near the capture probes on the array and therefore improve binding between the analytes and the capture probes, thus improving subsequent assay performance.

In some embodiments, at least one of the two semi-porous materials between the biological sample and the array can act as a molecular sieving matrix for electrophoretic analyte separation. For example, separation of analytes can occur based on physicochemical properties such as charge, size (e.g., length, radius of gyration, and effective diameters, etc.), electrophoretic mobility, zeta potential, isoelectric point, hydrophobicity, hydrophilicity, molecular binding (e.g., immunoaffinity), or any combination thereof. In some embodiments, at least one of the two semi-porous materials between the biological sample and the array can be of a uniform pore size. In some embodiments, at least one of the two semi-porous materials between the biological sample and the array can have discontinuities in pore size, as generally used in different gel electrophoresis schemes. In some embodiments, at least one of the two semi-porous materials between the biological sample and the array can have gradients in pore sizes. For example, at least one of the two semi-porous materials (e.g., a hydrogel or an organogel) can have a gradient of pore sizes such that the gradient separates the analytes as the analytes migrate to the array.

In some embodiments, different molecules in the biological sample migrate through at least one of the two semi-porous materials at different rates. For example, a smaller molecule can move through at least one of the two semi-porous materials at a faster rate relative to a larger molecule. In some embodiments, after applying the electric field for a period of time (e.g., any of the exemplary periods of time described herein), only one or more analytes (or one or more types of analytes) can move through at least one of the two semi-porous materials while other molecules (or types of molecules) are still in (retained in) at least one of the two semi-porous materials. In some embodiments, by controlling the amount of time the electric field is applied, one or more analytes can be selectively retained as they migrate through at least one of the two semi-porous materials. In some embodiments, the migration rate of the molecules can be controlled by varying the intensity of the electric field, or varying at least one of the two semi-porous materials. In some embodiments, by controlling the intensity of the electric field and the period of time the electric field is applied, one or more analytes can be selectively retained as they migrate through at least one of the two semi-porous materials. In some embodiments, the electric field is removed after the one or more analytes of interest have migrated through at least one of the two semi-porous materials. In some embodiments, at least one of the two semi-porous materials are removed after the one or more analytes of interest have moved through at least one of the two semi-porous materials.

In some embodiments, the first semi-porous material can retain one or more second analytes. In some embodiments, the first semi-porous material can retain one or more second analytes through specific binding. In some embodiments, the first semi-porous material contains a plurality of analyte capture agents (e.g., any of the exemplary analyte capture agents described herein) that include an analyte-binding moiety (e.g., any of the exemplary analyte-binding moieties described herein) and each analyte-binding moiety can specifically bind a second analyte of the one or more second analytes. In some embodiments, the analyte-binding moieties can be antibodies or antigen-binding fragments thereof (e.g., a Fab). In some embodiments, the one or more second analytes can be proteins. In some embodiments, the analyte-binding moieties can specifically bind certain nucleic acids (or classes or subclasses of nucleic acids). In some embodiments, the one or more second analytes are nucleic acids (or classes or subclasses of nucleic acids). In some embodiments, the first semi-porous material can be removed after it retains the one or more second analytes of interest. In some embodiments, the electric field is removed after the first semi-porous material retains the one or more second analytes of interest. In some embodiments, the analyte-binding moieties can be cross-linked to the first semi-porous material. In some embodiments, the analyte-binding moieties are cross-linked to the first semi-porous material via photoreactive cross-linkers (e.g., any of the exemplary photoreactive cross-linkers described herein).

In some embodiments, the first semi-porous material can retain one or more second analytes non-specifically. In some embodiments, the first semi-porous material can retain one or more second analytes based on physicochemical properties such as charge, size (e.g., length, radius of gyration, effective diameters, etc.), hydrophobicity/hydrophilicity, molecular binding (e.g., immunoaffinity), or any combination thereof. In some embodiments, the first semi-porous material can retain all molecules including analytes that are larger than a threshold molecular size (e.g., any of the exemplary threshold molecular sizes described herein). In some embodiments, the semi-porous material(s) can retain all nucleic acids that are longer than a threshold number of nucleotides (e.g., any of the exemplary threshold number of nucleotides described herein).

In some embodiments, different molecules in the biological sample move through the one semi-porous material(s) at different speeds. In some embodiments, after applying the electric field for a certain period of time (e.g., any of the exemplary periods of time described herein), one or more second analytes are retained in the first semi-porous material while other analytes have either migrated through the first semi-porous material and/or have yet to enter the first semi-porous material. In some embodiments, by controlling the period of time for which the electric field is applied, the one or more second analytes can be selectively retained in the first semi-porous material. In some embodiments, the rate of migration of analytes in the biological sample can be controlled by varying the intensity of the electric field that is applied to the biological sample, the first semi-porous material, and the array. In some embodiments, by controlling the intensity of the electric field and the period of time the electric field is applied, the one or more second analytes can be selectively retained in the first semi-porous material. In some embodiments, the electric field is removed after the first semi-porous material retains the one or more second analytes. In some embodiments, the first semi-porous material is removed after the first semi-porous material retains the one or more second analytes. In some embodiments, the first semi-porous material can retain one or more second analytes both non-specifically and through specific binding. In some embodiments, the first semi-porous material includes analyte-binding moieties (e.g., any of the analyte-binding moieties described herein) that can optionally be cross-linked to the first semi-porous material. In some embodiments, the analyte-binding moieties can be cross-linked to the first semi-porous material via photoreactive cross-linkers (e.g., any of the exemplary photoreactive cross-linkers described herein).

In some embodiments, the second semi-porous material can retain one or more third analytes (e.g., any of the types or classes of analytes described herein). In some embodiments, the second semi-porous material can retain one or more third analytes through specific binding. In some embodiments, the second semi-porous material contains a plurality of analyte capture agents which include an analyte binding moiety (e.g., any of the exemplary analyte binding moieties described herein) and each analyte-binding moiety can specifically bind a third analyte of the one or more third analytes. In some embodiments, the analyte-binding moieties can be antibodies or antigen-binding fragments thereof (e.g., a Fab). In some embodiments, the one or more third analytes can be proteins. In some embodiments, the analyte-binding moieties can specifically bind certain nucleic acids or a subclass of nucleic acids. In some embodiments, the one or more third analytes are nucleic acids or a subclass of nucleic acids. In some embodiments, the second semi-porous material is removed after it retains the one or more third analytes of interest. In some embodiments, the electric field is removed after the second semi-porous material retains the one or more third analytes of interest. In some embodiments, the analyte-binding moieties can be cross-linked to the second semi-porous material. In some embodiments, the analyte-binding moieties can be cross-linked to the second semi-porous material via photoreactive cross-linkers (e.g., any of the exemplary photoreactive cross-linkers described herein).

In some embodiments, the second semi-porous material can retain one or more third analytes non-specifically. In some embodiments, the second semi-porous material can retain one or more third analytes based on physicochemical properties such as charge, size (e.g., length, radius of gyration, effective diameters, etc.), hydrophobicity/hydrophilicity, molecular binding (e.g., immunoaffinity), or a combination thereof. In some embodiments, the second semi-porous material can retain all analytes that are larger than a threshold molecular size (e.g., any of the exemplary threshold molecular sizes described herein). In some embodiments, the second semi-porous material can retain all nucleic acid analytes that are longer than a threshold length of nucleotides (e.g., any of the exemplary thresholding lengths of nucleic acids described herein). In some embodiments, different molecules in the biological sample move through the second semi-porous material at different rates. In some embodiments, after applying the electric field for a certain period of time, one or more third analytes are retained in the second semi-porous material while other analytes have either migrated through the second semi-porous material and/or have yet to enter the second semi-porous material. In some embodiments, controlling the period of time the electric field is applied, the one or more third analytes can be selectively retained in the second semi-porous material. In some embodiments, the migration rate of the molecules in the biological sample can be controlled by varying the intensity of the electric field that is applied to the biological sample, the second semi-porous material, and the array. In some embodiments, by controlling the intensity of the electric field and the period of time the electric field is applied, the one or more third analytes can be selectively retained in the second semi-porous material. In some embodiments, the electric field is removed after the second semi-porous material retains the one or more third analytes of interest. In some embodiments, the second semi-porous material is removed after the second semi-porous material retains the one or more third analytes of interest. In some embodiments, the second semi-porous material can retain one or more third analytes both non-specifically and through specific binding. In some embodiments, the second semi-porous material can include analyte-binding moieties (e.g., analyte-binding moieties that are cross-linked to the second semi-porous material). In some embodiments, the analyte-binding moieties can be cross-linked to the second semi-porous material via photoreactive cross-linkers (e.g., any of the exemplary photoreactive cross-linkers described herein).

In some embodiments, only one of the two semi-porous materials contain analyte capture agents (e.g., any of the exemplary analyte capture agents described herein) which include an analyte binding moiety (e.g., any of the exemplary analyte binding moieties described herein). In some embodiments, only the second semi-porous materials contains analyte-binding moieties that can specifically bind to one or more third analytes. In some embodiments, the first semi-porous material can retain one or more second analytes non-specifically and the second semi-porous material can retain one or more third analytes specifically. In some embodiments, the first semi-porous material can retain one or more second analytes based on the size of the analytes and the second semi-porous material contains antibodies or antigen-binding fragments thereof (e.g., a Fab) that can bind specifically to one or more third analytes.

In some embodiments, both the first and the second semi-porous materials contain analyte capture agents (e.g., any of the exemplary analyte capture agents described herein) which include analyte-binding moieties (e.g., any of the exemplary analyte binding moieties described herein). In some embodiments, the first semi-porous material contains a first type of analyte capture agent and the second semi-porous material contains a second type of analyte capture agent. In some embodiments, the first semi-porous material contains analyte capture agents (e.g., such as antibodies) and the second semi-porous material contains analyte capture agents that can specifically bind certain nucleic acids. In some embodiments, the first semi-porous material retains a protein and the second semi-porous material retains a type, class, or a subclass of nucleic acids. In some embodiments, the first semi-porous material contains a first analyte capture agent (e.g., a first antibody) and the second semi-porous material contains a second analyte capture agent (e.g., a second antibody). In some embodiments, the first semi-porous material retains a first protein and the second semi-porous material retains a second protein.

In some embodiments, neither the first nor the second semi-porous material contains any analyte capture agents (e.g., any of the analyte capture agents described herein). In some embodiments, the first semi-porous material can retain one or more second analytes based on the size of the analytes and the second semi-porous material can retain one or more third analytes based on the charge of the analytes. In some embodiments, the first semi-porous material can retain one or more second analytes based on the size of the analytes and the second semi-porous material can retain one or more third analytes based on the size of the analytes. In some embodiments, the first semi-porous material can retain analytes larger than a first threshold size (e.g., any of the exemplary threshold sizes described herein) and the second semi-porous material can retain all molecules larger than a second threshold size (e.g., any of the exemplary threshold sizes described herein). In some embodiments, the first threshold size is greater than the second threshold size.

In some embodiments, the methods described herein can further include crosslinking the one or more second analytes to the second semi-porous material. In some embodiments, the methods described herein can further include crosslinking the one or more second analytes to the second semi-porous material via photoreactive cross-linkers (e.g., any of the exemplary photoreactive cross-linkers described herein). In some embodiments, the methods described herein can further include crosslinking the one or more third analytes to the second semi-porous material. In some embodiments, the methods can further include crosslinking the one or more third analytes to the second semi-porous material via photoreactive cross-linkers (e.g., any of the exemplary photoreactive cross-linkers described herein).

In some embodiments, the methods described herein can include the use of a third semi-porous material (e.g., a third semi-porous material that retains one or more fourth analytes). In some embodiments, the methods described herein can include the use of a fourth semi-porous material (e.g., a fourth semi-porous material that retains one or more fifth analytes).

e) Determining the Location of the One or More Analytes

In some embodiments, one or more first analytes pass through the first and second semi-porous materials and bind (e.g., hybridize) to the capture domains on the capture probes on the array. In some embodiments, one or more mRNAs pass through the first and the second semi-porous materials and bind (e.g., hybridize) to the capture domains on the capture probes on the array. In some embodiments, all or a portion of the sequence of the capture probes (e.g., the spatial barcodes of the capture probes) on the array, or complement thereof, and all or a portion of the sequence of the corresponding captured first analytes, or complement thereof, are determined. In some embodiments, the location of the one or more first analytes in the biological sample are determined based on all or a portion of the sequence of the capture probes (e.g., the spatial barcodes of the capture probes) on the array, or a complement thereof, and all or a portion of the sequence of the corresponding captured one or more first analytes, or a complement thereof. In some embodiments, determining the location of the one or more first analytes in the biological sample includes determining all or a portion of the sequence of the capture probes (e.g., the spatial barcodes of the capture probes) on the array, or a complement thereof, and all or a portion of the sequence of the corresponding captured one or more first analytes, or a complement thereof, and correlating such sequence information to an image of the biological sample. Some embodiments of the methods described herein further include obtaining an image of the biological sample.

In some embodiments, the first semi-porous material retains one or more second analytes. In some embodiments, the location of the one or more second analytes that are retained in the first semi-porous material is determined by immunofluorescence. In some embodiments, one or more detectable labels (e.g., fluorophore-labeled antibodies) bind to the one or more second analytes that are retained in the first semi-porous material and the location of the one or more second analytes is determined by detecting the detectable labels under suitable conditions. In some embodiments, one or more fluorophore-labeled antibodies bind to the one or more second analytes that are retained in the first semi-porous material and the location of the one or more second analytes is determined by imaging the fluorophore-labeled antibodies when the fluorophores are excited by a light of a suitable wavelength. In some embodiments, the location of the one or more second analytes in the biological sample is determined by correlating the immunofluorescence data to the image of the biological sample. In some embodiments, one or more nucleic acids are retained in the first semi-porous material. In some embodiments, the location of the one or more second analytes that are retained in the first semi-porous material is determined by in situ hybridization. In some embodiments, the location of the one or more second analytes in the biological sample is determined by correlating the in situ hybridization data to the image of the biological sample.

In some embodiments, the second semi-porous material retains one or more third analytes (e.g., any of the classes or subclass of analytes described herein). In some embodiments, the location of the one or more third analytes that are retained in the second semi-porous material is determined by immunofluorescence. In some embodiments, one or more detectable labels (e.g., fluorophore-labeled antibodies) bind to the one or more third analytes that are retained in the second semi-porous material and the location of the one or more third analytes is determined by detecting the detectable labels under suitable conditions. In some embodiments, one or more fluorophore-labeled antibodies bind to the one or more third analytes that are retained in the second semi-porous material and the location of the one or more third analytes is determined by imaging the fluorophore-labeled antibodies when the fluorophores are excited by a light of a suitable wavelength. In some embodiments, the location of the one or more third analytes in the biological sample is determined by correlating the immunofluorescence data to an image of the biological sample. In some embodiments, one or more nucleic acids are retained in the second semi-porous material. In some embodiments, the location of the one or more third analytes that are retained in the second semi-porous material is determined by in situ hybridization. In some embodiments, the location of the one or more third analytes in the biological sample is determined by correlating the in situ hybridization data to an image of the biological sample.

In some embodiments, the first semi-porous material retains one or more second analytes from the biological sample and the second semi-porous material retains one or more third analytes from the biological sample. In some embodiments, the location of the one or more second analytes in the first semi-porous material is determined by immunofluorescence and the location of the one or more third analytes in the second semi-porous material is determined by in situ hybridization. In some embodiments the location of the one or more second analytes is determined by correlating the immunofluorescence data to an image of the biological sample, and the location of the one or more third analytes is determined by correlating the in situ hybridization data to an image of the biological sample.

In some embodiments, the methods described herein can further include providing a second array that contains a plurality of second capture probes. In some embodiments, each second capture probe contains (i) a second spatial barcode and (ii) a second capture domain that can bind (e.g., hybridize) to one or more second analytes or an analyte capture sequence. In some embodiments, the methods described herein can further include applying a second electric field to the first semi-porous material containing the one or more second analytes and the second array so that the electric field promotes the migration of the one or more second analytes in the direction of the second array. In some embodiments, the one or more second analytes bind (e.g., hybridize) to the second capture domains on the second capture probes on the second array. In some embodiments, the one or more second analytes are miRNAs. In some embodiments, all or a portion of the sequence of the second capture probes (e.g., sequence of all or a part of the spatial barcodes) on the second array, or a complement thereof, and all or a portion of the sequence of the corresponding captured second analytes, or a complement thereof, are determined. In some embodiments, the location of the one or more second analytes in the biological sample are determined based on all or a portion of the sequence of the second capture probes (e.g., the sequence of all or a portion of the spatial barcodes) on the second array, or a complement thereof, and all or a portion of the sequence of the corresponding captured one or more second analytes, or a complement thereof. In some embodiments, determining the location of the one or more second analytes in the biological sample includes determining all or a portion of the sequence of the second capture probes (e.g., all or a portion of the spatial barcodes) on the second array, or a complement thereof, and all or a portion of the sequence of the corresponding captured one or more second analytes, or a complement thereof, and correlating such sequence information to an image of the biological sample. In some embodiments, the methods described herein further include obtaining an image of the biological sample.

In some embodiments, the methods described herein can further include providing a third array that contains a plurality of third capture probes. In some embodiments, each third capture probe contains (i) a third spatial barcode and (ii) a third capture domain that can bind (e.g., hybridize) to one or more third analytes. In some embodiments, the methods described herein can further include applying a third electric field to the second semi-porous material containing the one or more third analytes and the third array so that the electric field promotes the migration of the one or more third analytes in the direction of the third array. In some embodiments, the one or more third analytes bind (e.g., hybridize) to the third capture domains on the third capture probes on the third array. In some embodiments, the one or more third analytes can be miRNAs. In some embodiments, all or a portion of the sequence of the third capture probes (e.g., all or a portion of the spatial barcodes of the third capture probes) on the third array, or a complement thereof, and all or a portion of the sequence of the corresponding captured third analytes, or a complement thereof, are determined. In some embodiments, the location of the one or more third analytes in the biological sample are determined based on all or a portion of the sequence of the third capture probes (e.g., all or a portion of the spatial barcodes of the third capture probes) on the third array, or a complement thereof, and all or a portion of the sequence of the corresponding captured one or more third analytes, or a complement thereof. In some embodiments, determining the location of the one or more third analytes in the biological sample includes determining all or a portion of the sequence of the third capture probes (e.g., all or a portion of the spatial barcodes of the third capture probes) on the third array, or a complement thereof, and all or a portion of the sequence of the corresponding captured one or more third analytes, or a complement thereof, and correlating such sequence information to an image of the biological sample.

f) Analyte Capture Agent

In some embodiments, the methods described herein include providing a first array that contains a plurality of first capture probes. In some embodiments, each first capture probe contains (i) a first spatial barcode and (ii) a first capture domain. In some embodiments, the methods described herein can further include providing a second array that contains a plurality of second capture probes. In some embodiments, each second capture probe contains (i) a second spatial barcode and (ii) a second capture domain that can bind to one or more analyte capture sequences.

In some embodiments, the methods described herein can further include providing one semi-porous material between the biological sample and the array. In some embodiments, the semi-porous material can be a permeable membrane. In some embodiments, the semi-porous material can be a gel (e.g., a hydrogel or an organogel). For example, the semi-porous material can be a SDS-PAGE gel. In some embodiments, the semi-porous material can have a substantially uniform pore size. In some embodiments, the semi-porous material can have non-uniform pore sizes. For example, the semi-porous material can be a gradient gel. In some embodiments, the semi-porous material can contain a permeabilization buffer. In some embodiments, the semi-porous material can contain an electrophoresis buffer.

In some embodiments, the semi-porous material contains a plurality of analyte capture agents (e.g., any of the exemplary analyte capture agents described herein) and each analyte capture agent contains (i) an analyte binding moiety that can specifically bind to the second analyte (e.g., any of the exemplary analyte binding moieties described herein), (ii) analyte binding moiety barcode and (iii) an analyte capture sequence that can specifically bind (e.g., hybridize) to the second capture domain of the second capture probe on the second array. In some embodiments, the analyte binding moiety can be an antibody or an antigen-binding fragment (e.g., a Fab) thereof. In some embodiments, the one or more second analytes can be proteins. In some embodiments, the semi-porous material can retain one or more second analytes through the use of the analyte capture agents (e.g., via specific binding through the analyte binding moiety). In some embodiments, the analyte capture agents can be released from the semi-porous material through the use of a cleavage domain.

In some embodiments, methods for determining the location of analytes in a biological sample include applying a first electric field to the biological sample, the semi-porous material and the first array so that the first electric field promotes the migration of one or more analytes (e.g., one or more first analytes) in the direction of the first array. In some embodiments, one or more first analytes pass through the semi-porous material and bind (e.g., hybridize) to the first capture domains on the capture probes on the first array. In some embodiments, one or more mRNAs pass through the semi-porous material and bind (e.g., hybridize) to the first capture domains on the first capture probes on the first array. In some embodiments, all or a portion of the sequence of the first capture probes (e.g., all or a portion of the spatial barcodes of the first capture probes) on the first array, or a complement thereof, and all or a portion of the sequence of the corresponding captured first analytes, or a complement thereof, are determined. In some embodiments, the location of the one or more first analytes in the biological sample are determined based on all or a portion of the sequence of the first capture probes (e.g., all or a portion of the spatial barcodes of the first capture probes) on the first array, or a complement thereof, and all or a portion of the sequence of the corresponding captured one or more first analytes, or a complement thereof. In some embodiments, determining the location of the one or more first analytes in the biological sample includes determining all or a portion of the sequence of the first capture probes (e.g., all or a portion of the spatial barcodes of the first capture probes) on the first array, or a complement thereof, and all or a portion of the sequence of the corresponding captured one or more first analytes, or a complement thereof, and correlating such sequence information to an image of the biological sample.

In some embodiments, the semi-porous material can retain one or more second analytes through the use of the analyte capture agents. In some embodiments, after the semi-porous material retains one or more second analytes, the semi-porous material is removed. In some embodiments, after the semi-porous material retains one or more second analytes, the first electric field is removed. In some embodiments, the second analytes bound to the analyte capture agents are released from the semi-porous material through the use of a cleavage domain in the analyte capture agents.

In some embodiments, the methods described herein can further include applying a second electric field to the semi-porous material containing the one or more second analytes and the second array so that the electric field promotes the migration of the one or more second analytes in the direction of the second array. In some embodiments, the one or more second analytes bind to the second array through specific binding (e.g., hybridization) between the analyte capture sequence of the analyte capture agents and the second capture domain on the second capture probes on the second array. In some embodiments, the one or more second analytes are proteins. In some embodiments, all or a portion of the sequence of the second capture probes (e.g., all or a portion of the spatial barcodes of the second capture probes) on the second array, or a complement thereof, and all or a portion of the sequence of the corresponding analyte binding moiety barcodes, or a complement thereof, on the captured analyte capture agents are determined. In some embodiments, the location of the one or more second analytes in the biological sample are determined based on all or a portion of the sequence of the second capture probes (e.g., all or a portion of the spatial barcodes of the second capture probes) on the second array, or a complement thereof, and all or a portion of the sequence of the corresponding analyte binding moiety barcodes, or a complement thereof, on the captured analyte capture agents. In some embodiments, determining the location of the one or more second analytes in the biological sample includes determining all or a portion of the sequence of the second capture probes (e.g., all or a portion of the spatial barcodes of the second capture probes) on the second array, or a complement thereof, and all or a portion of the sequence of the corresponding analyte binding moiety barcodes, or a complement thereof, on the captured analyte capture agents and correlating such sequence information to an image of the biological sample.

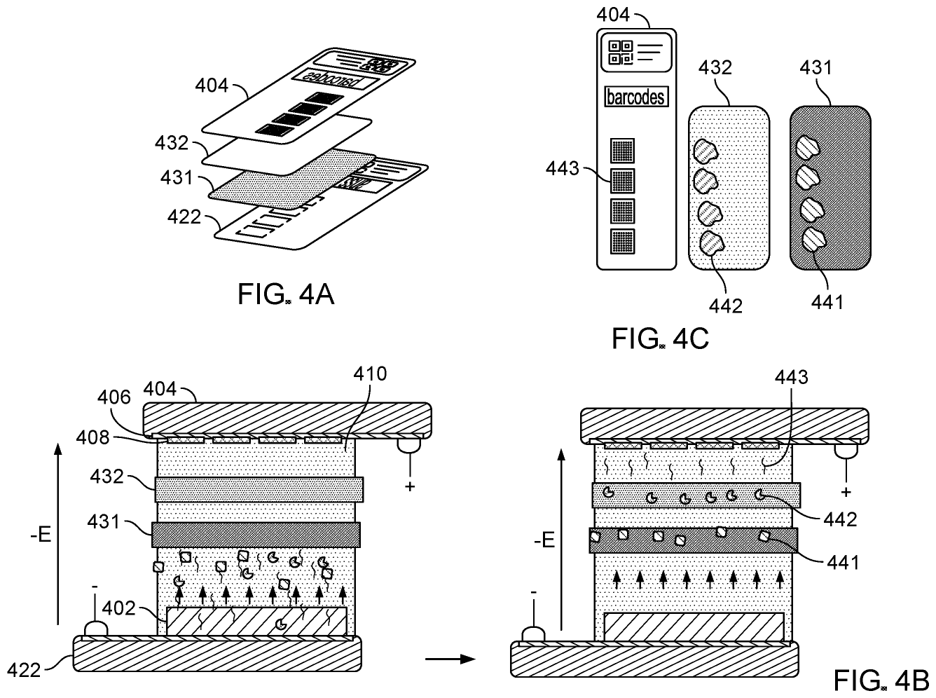

FIGS. 4A-C shows an example of spatially transferring analytes and multi-omic spatial analysis. FIG. 4A shows an example substrate configuration for use in spatially transferring analytes from a biological sample to an array of capture probes and two semi-porous materials. In some examples, a biological sample 402 (e.g., a tissue sample) can be in contact with a second substrate 422. In some embodiments, the biological sample 402 contains three analytes of interest 441-43. In some embodiments, first substrate 404 can have one or more coatings (e.g., any of the conductive substrates described herein) on its surface. Non-limiting examples of coatings include conductive oxides (e.g., indium tin oxide). In some embodiments, first substrate 404 can have a functionalization chemistry on its surface. In the examples shown in FIGS. 4A-C, first substrate 404 is overlaid with a first coating 406, and first coating 406 (e.g., a conductive coating) is further overlaid with capture probes 408. In some embodiments, first coating 406 is an indium tin oxide (ITO) coating. In some embodiments, capture probes 408 are an array of capture probes (e.g., any of the capture probes described herein). In some embodiments, a substrate can include an ITO coating. In some embodiments, a substrate can include capture probes or capture probes attached to features on the substrate. In some embodiments, two semi-porous materials 431-32 are placed between the first substrate 404 and the second substrate 422. In some embodiments, one or more of the two semi-porous materials 431-32 are hydrogels. In some embodiments, one or more of the two semi-porous materials 431-32 are nitrocellulose films. In some embodiments, one or more of the two semi-porous materials 431-32 are functionalized with chemistries to specifically or non-specifically retain analytes (e.g., antibody functionalization to specifically capture protein analytes, specific oligo probe functionalization to specifically retain microRNA analytes).

FIG. 4B shows an example schematic workflow for spatially transferring analytes 441-443. In some embodiments, biological sample 402 includes three analytes 441-443. In some embodiments, the analytes 441-443 are negatively charged. First substrate 404 can include an array of capture probes 408 that is fixed or attached to the first substrate 404 or attached to features (e.g., beads) on the substrate. In some embodiments, capture probes 408 can include any of the capture probes disclosed herein. In some embodiments, first substrate 402 does not include features and instead, capture probes 408 are directly attached to the substrate surface. In some embodiments, the capture probes 408 are positively charged.

Biological sample 402, the two semi-porous materials 431-32 and capture probes 408 (e.g., an array of capture probes) can be in contact with a buffer 410. In some embodiments, buffer 410 can include a permeabilization reagent. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include but are not limited to dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 441-43 are proteins or nucleic acids. In some embodiments, the analytes 441-43 are negatively charged proteins or nucleic acids. In some embodiments, the capture probes 408 are proteins or nucleic acids. In some embodiments, the capture probes 408 are positively charged proteins or nucleic acids. In some embodiments, the analytes 443 are negatively charged transcripts. In some embodiments, the analytes 443 are poly(A) containing transcripts. In some embodiments, the capture probes 408 are attached to a feature in a feature array. In some embodiments, buffer 410 can be in contact with the biological sample 402, first substrate 404, second substrate 422, or any combination thereof.

In FIG. 4B, the biological sample 402 can be subjected to electrophoresis. During electrophoresis, the biological sample 402 is subjected to an electric field that can be generated by sandwiching biological sample 402 between the first substrate 404 and a second substrate 422, connecting each substrate to a cathode and an anode, respectively, and running an electric current through the substrates. The application of the electric field “-E” causes the analytes 441-443 (e.g., negatively charged analytes) to migrate towards the substrate 404 and capture probes 408 (e.g., positively charged capture probes) in the direction of the arrows shown in FIG. 4B. In some embodiments, the analytes 441-443 migrate towards capture probes 408 through one or more permeabilized cells within the permeabilized biological sample (e.g., from an original location in a permeabilized cell to a final location in or close to the capture probes 408). Second substrate 422 can include the first coating 406 (e.g., a conductive coating), thereby allowing electric field “-E” to be generated. In some embodiments, the analyte 441 is retained by the semi-porous material 431. In some embodiments, the analyte 442 is retained by the semi-porous material 432. In some embodiments, the analyte 443 is captured by the capture probes 408 on the first substrate 404.

FIG. 4C shows the results of spatially transferring the analytes 441-43 according to the configuration in FIG. 4A. FIG. 4B shows that the analyte 441 is retained by the semi-porous material 441; the analyte 442 is retained by the semi-porous material 442; and the analyte 443 is captured by the capture probes 408 on the first substrate 404. In some embodiments, analyte 441 is a protein; analyte 442 is a microRNA; and analyte 443 is a mRNA. In some embodiments, after spatially transferring the analytes 441-43, the semi-porous material 431 containing analyte 441 is subject to immunofluorescence analysis. In some embodiments, after spatially transferring the analytes 441-43, the semi-porous material 432 containing analyte 442 is subject to an in situ hybridization analysis. In some embodiments, after spatially transferring the analytes 441-43, the sequence of the capture probes 408 (or complement thereof) and the sequence of analyte 443 (or complement thereof) are determined.

### Other Embodiments

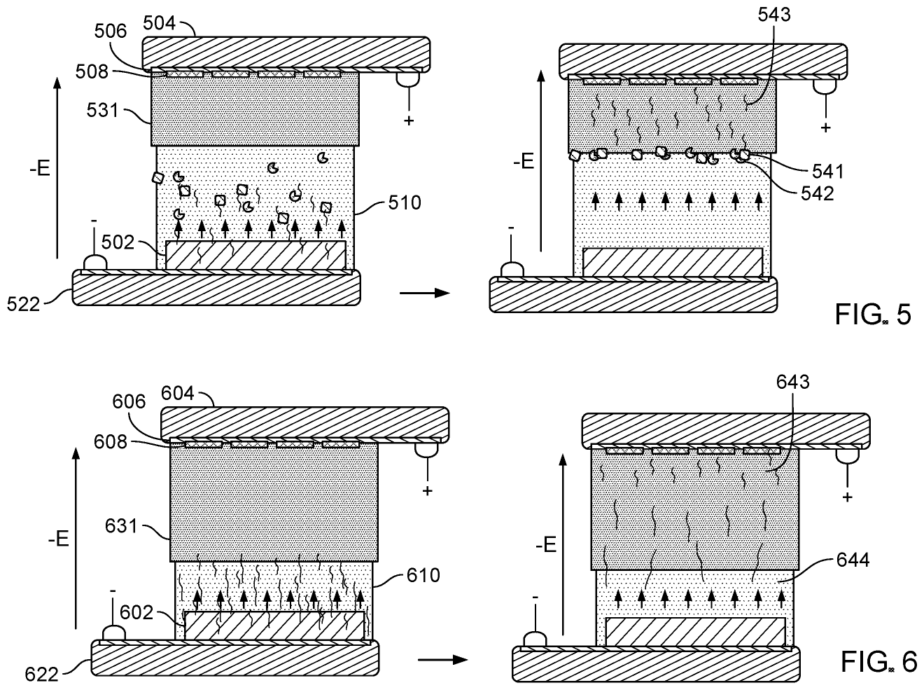

FIG. 5 shows an example schematic workflow for spatially transferring analytes 541-543, where the semi-porous material 531 is used as a filter. In some embodiments, a gel or other type of semi-porous material 531 can be used to act as a filter to exclude molecules other than the target analytes 543. In this example, RNA transcripts are the target analyte 543 that can pass through the filter 531 and other molecules such as proteins, lipids, etc. are excluded. In some embodiments, the selection of excluded molecules can be based on physicochemical properties such as charge, size (length, radius of gyration, effective diameters, etc.), hydrophobicity, hydrophilicity, molecular binding (e.g., immunoaffinity), etc. In some embodiments, using the semi-porous material 531 as a filter can reduce non-specific binding of the capture probes 508 and therefore improve binding specificity and assay performance.

In some embodiments, biological sample 502 includes three analytes 541-543. In some embodiments, the analytes 541-543 are charged (e.g., a positive or a negative charge). First substrate 504 can include an array of capture probes 508 that are fixed or attached to the first substrate 504 or attached to features (e.g., beads or wells or locations) on the substrate. In some embodiments, capture probes 508 can include any of the capture probes disclosed herein. In some embodiments, first substrate 504 does not include features and instead, capture probes 508 are directly attached to the substrate surface. In some embodiments, the capture probes 508 are positively charged.

Biological sample 502, a semi-porous material 531 and capture probes 508 (e.g., an array of capture probes) can be in contact with a buffer 510. In some embodiments, buffer 510 can include a permeabilization reagent. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include, but are not limited to, dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 541-43 are proteins and/or nucleic acids. In some embodiments, the analytes 541-43 are negatively charged proteins or nucleic acids. In some embodiments, the capture probes 508 are proteins and/or nucleic acids. In some embodiments, the capture probes 508 are positively charged proteins and/or nucleic acids. In some embodiments, the analytes 543 are negatively charged transcripts. In some embodiments, the analytes 543 are poly(A) containing transcripts. In some embodiments, the capture probes 508 are attached to a feature on an array. In some embodiments, buffer 510 can be in contact with the biological sample 502, first substrate 504, second substrate 522, or any combination thereof.

In FIG. 5, the biological sample 502 can be subjected to electrophoresis. During electrophoresis, the biological sample 502 is subjected to an electric field that can be generated by placing the biological sample 502 between the first substrate 504 and a second substrate 522, connecting each substrate to a cathode and an anode, respectively, and running an electric current through the substrates. The application of the electric field “-E” causes the analytes 541-543 (e.g., negatively charged analytes) to migrate towards the substrate 504 and capture probes 508 (e.g., positively charged capture probes) in the direction of the arrows shown in FIG. 5. In some embodiments, the analytes 541-543 migrate towards capture probes 508 from the permeabilized biological sample (e.g., from an original location in a permeabilized cell to a final location in or close to the capture probes 508). Second substrate 522 can include the first coating 506 (e.g., a conductive coating), thereby allowing electric field “-E” to be generated. In some embodiments, the analytes 541-542 are retained by the semi-porous material 531. In some embodiments, the analyte 543 is captured by the capture probes 508 on the first substrate 504.

FIG. 6 shows an example schematic workflow for spatially transferring analytes 643-644, where the semi-porous material 631 is used as a molecular sieve. In some embodiments, a gel or other type of semi-porous material 631 can be used to act as a molecular sieve for electrophoretic molecular separation. The separation can occur based on physicochemical properties such as charge, size (length, radius of gyration, effective diameters, etc.), electrophoretic mobility, zeta potential, isoelectric point, hydrophobicity, hydrophilicity, molecular binding (e.g., immunoaffinity), etc. In some embodiments, the semi-porous material 631 can be of a uniform pore size. In some embodiments, the semi-porous material 631 can have discontinuities in pore size, or gradients in pore size, as generally used in different gel electrophoresis schemes. In the example shown in FIG. 6, analytes 643-644 are separated based on the length of the analytes, with shorter analytes 643 having a higher electrophoretic mobility thus migrating further through the semi-porous material 631 compared to longer analytes 644. In some embodiments, specific sub-fractions of analytes 643-644 can be selected by using different semi-porous material 631 compositions and different electrophoresis schemes (e.g., different duration or voltage).

In some embodiments, biological sample 602 includes analytes 643-644. In some embodiments, the analytes 643-644 are negatively charged. First substrate 604 can include an array of capture probes 608 that are fixed or attached to the first substrate 604 or attached to features (e.g., beads) on the substrate. In some embodiments, capture probes 608 can include any of the capture probes disclosed herein. In some embodiments, first substrate 602 does not include features and instead, capture probes 608 are directly attached to the substrate surface.

Biological sample 602, a semi-porous material 631 and capture probes 608 (e.g., an array of capture probes) can be in contact with buffer 610. In some embodiments, buffer 610 can contain a permeabilization buffer. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include, but are not limited to, dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 643-644 can be proteins and/or nucleic acids. In some embodiments, the analytes 643-644 are negatively charged proteins and/or nucleic acids. In some embodiments, the capture probes 608 are proteins and/or nucleic acids. In some embodiments, the capture probes 608 are positively charged proteins and/or nucleic acids. In some embodiments, the analyte 643-644 are negatively charged transcripts. In some embodiments, the analyte 643-644 are poly(A) containing transcripts. In some embodiments, the capture probes 608 are attached to a feature in a feature array. In some embodiments, buffer 610 can be in contact with the biological sample 602, first substrate 604, second substrate 622, or any combination thereof.

In FIG. 6, the biological sample 602 can be subjected to electrophoresis. During electrophoresis, the biological sample 602 is subjected to an electric field that can be generated by placing the biological sample 602 between the first substrate 604 and a second substrate 622, connecting each substrate to a cathode and an anode, respectively, and running an electric current through the substrates. The application of the electric field “-E” causes the analytes 643-644 (e.g., negatively charged analytes) to migrate towards the substrate 604 and capture probes 608 in the direction of the arrows shown in FIG. 6. In some embodiments, the analytes 643-644 migrate towards capture probes 608 from the permeabilized biological sample (e.g., from an original location in a permeabilized cell to a final location in or close to the capture probes 608). Second substrate 622 can include the first coating 606 (e.g., a conductive coating), thereby allowing electric field “-E” to be generated. In some embodiments, shorter analytes 643 migrate more efficiently through the semi-porous material 631 and reach the capture probes 608 while longer analytes 644 are either excluded at the semi-porous material 631 interface or migrate more slowly through the semi-porous material 631 and do not reach the capture probes 608.

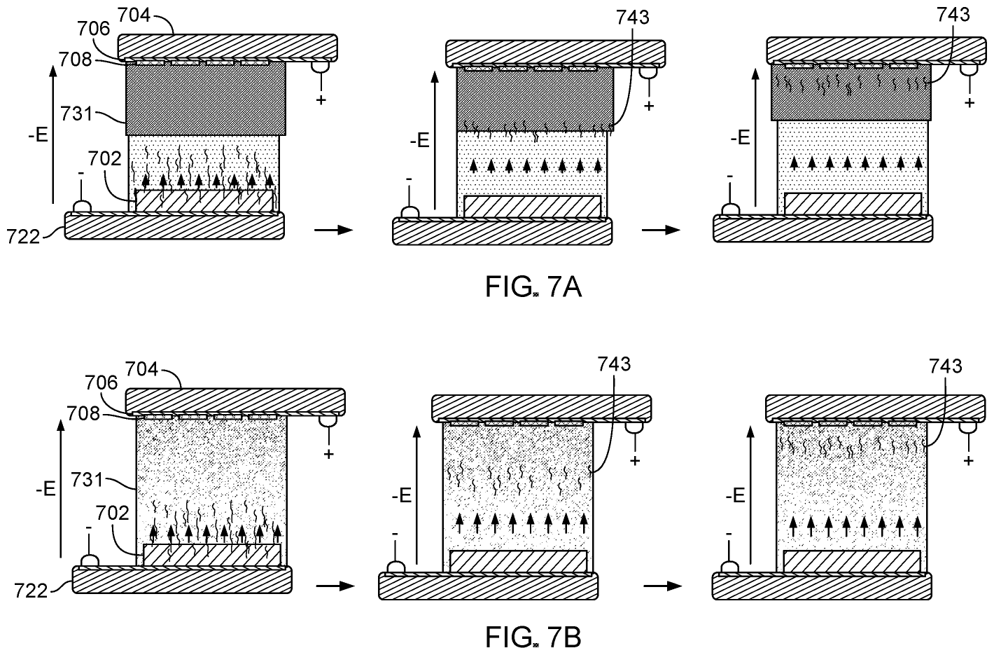

FIG. 7A-B show example schematic workflows for spatially transferring analytes 743, where the semi-porous material 731 creates molecular “stacking” (e.g., increasing the concentration of electro-migrating entities). In some embodiments, the semi-porous material 731 has discontinuities in pore size. In the example shown in FIG. 7A, the semi-porous material 731 has a smaller pore size in the part proximate to the capture probes 708 and a larger pore size in the part proximate to the biological sample 702. In some embodiments, analytes 743 slow down and stack as they migrate through the semi-porous material 731 due to the pore size discontinuity. In some embodiments, the semi-porous material 731 has a pore size gradient. In the example shown in FIG. 7B, the semi-porous material 731 has a pore size gradient with smaller pore sizes near the capture probes 708 and larger pore sizes near the biological sample 702. In some embodiments, analytes 743 decrease the rate of transfer and stack as they migrate through the semi-porous material 731 due to the pore size gradient. In some embodiments, this “stacking” effect increases the concentration of analytes 743 when they arrive at the capture probes 708, leading to favorable binding kinetics and better assay sensitivity. In some embodiments, pore size discontinuities can also be used to increase the separation resolution between molecules of different sizes and/or lengths.

In some embodiments, biological sample 702 includes analytes 743. In some embodiments, the analytes 743 are negatively charged. First substrate 704 can include an array of capture probes 708 that are fixed or attached to the first substrate 704 or attached to features (e.g., beads) on the substrate. In some embodiments, capture probes 708 can include any of the capture probes disclosed herein. In some embodiments, first substrate 702 does not include features and instead, capture probes 708 are directly attached to the substrate surface.

Biological sample 702, a semi-porous material 731 and capture probes 708 (e.g., an array of capture probes) can be in contact with buffer 710. In some embodiments, buffer 710 can contain a permeabilization buffer. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include but are not limited to dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 743 can be proteins and/or nucleic acids. In some embodiments, the analytes 743 are negatively charged proteins and/or nucleic acids. In some embodiments, the capture probes 708 are proteins and/or nucleic acids. In some embodiments, the capture probes 708 are positively charged proteins and/or nucleic acids. In some embodiments, the analyte 743 are negatively charged transcripts. In some embodiments, the analyte 743 are poly(A) containing transcripts. In some embodiments, the capture probes 708 are attached to a feature in a feature array. In some embodiments, buffer 710 can be in contact with the biological sample 702, first substrate 704, second substrate 722, or any combination thereof.

In FIG. 7A-B, the biological sample 702 can be subjected to electrophoresis. During electrophoresis, the biological sample 702 is subjected to an electric field that can be generated by sandwiching biological sample 702 between the first substrate 704 and a second substrate 722, connecting each substrate to a cathode and an anode, respectively, and running an electric current through the substrates. The application of the electric field “-E” causes the analytes 743 (e.g., negatively charged analytes) to migrate towards the substrate 704 and capture probes 708 in the direction of the arrows shown in FIG. 7A-B. In some embodiments, the analytes 743 migrate towards capture probes 708 from the permeabilized biological sample (e.g., from an original location in a permeabilized cell to a final location in or close to the capture probes 708). Second substrate 722 can include the first coating 706 (e.g., a conductive coating), thereby allowing electric field “-E” to be generated.

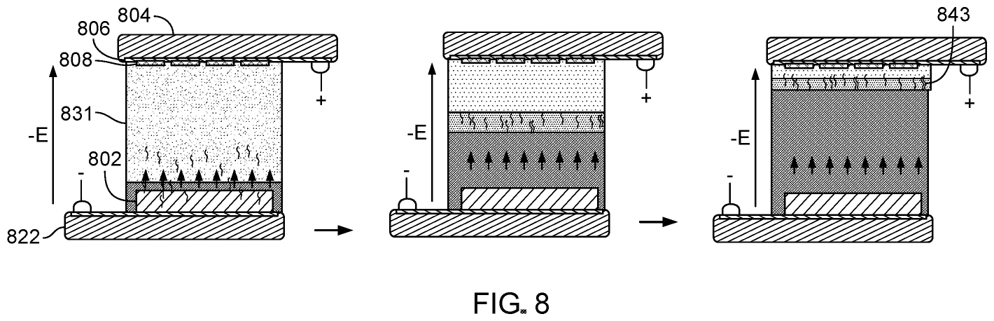

FIG. 8 shows an example schematic workflow for spatially transferring analytes 843, where the semi-porous material 831 creates molecular “stacking” (increasing the concentration of electro-migrating entities). In some embodiments, discontinuous buffers can be used for isotachophoresis (ITP). In the example shown in FIG. 8, the electrophoretic mobility of the leading electrolyte and trailing electrolyte buffers are chosen to stack the target analytes 843. In some embodiments, the analytes 843 are concentrated as they electro-migrate through the semi-porous material 831. In some embodiments, this “stacking” effect increases the concentration of analytes 843 when they arrive at the capture probes 808, leading to favorable binding kinetics and better assay sensitivity. In some embodiments, pore size discontinuities can also be used to increase the separation resolution between molecules of different sizes/lengths. In some embodiments, ITP can be used in conjunction with gel-based separations (e.g., ITP stacking before size-based separations).

In some embodiments, biological sample 802 includes analytes 843. In some embodiments, the analytes 843 are negatively charged. First substrate 804 can include an array of capture probes 808 that are fixed or attached to the first substrate 804 or attached to features (e.g., beads) on the substrate. In some embodiments, capture probes 808 can include any of the capture probes disclosed herein. In some embodiments, first substrate 802 does not include features and instead, capture probes 808 are directly attached to the substrate surface. In some embodiments, the capture probes 808 are positively charged.

Biological sample 802, a semi-porous material 831 and capture probes 808 (e.g., an array of capture probes) can be in contact with buffer 810. In some embodiments, buffer 810 can contain a permeabilization buffer. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include, but are not limited to, dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 843 can be proteins and/or nucleic acids. In some embodiments, the analytes 843 are negatively charged proteins and/or nucleic acids. In some embodiments, the capture probes 808 are proteins and/or nucleic acids. In some embodiments, the capture probes 808 are positively charged proteins and/or nucleic acids. In some embodiments, the analyte 843 are negatively charged transcripts. In some embodiments, the analyte 843 are poly(A) containing transcripts. In some embodiments, the capture probes 808 are attached to a feature in a feature array. In some embodiments, buffer 810 can be in contact with the biological sample 802, first substrate 804, second substrate 822, or any combination thereof.

In FIG. 8, the biological sample 802 can be subjected to electrophoresis. During electrophoresis, the biological sample 802 is subjected to an electric field that can be generated by sandwiching biological sample 802 between the first substrate 804 and a second substrate 822, connecting each substrate to a cathode and an anode, respectively, and running an electric current through the substrates. The application of the electric field “-E” causes the analytes 843 (e.g., negatively charged analytes) to migrate towards the substrate 804 and capture probes 808 in the direction of the arrows shown in FIG. 8. In some embodiments, the analytes 843 migrate towards capture probes 808 from the permeabilized biological sample (e.g., from an original location in a permeabilized cell to a final location in or close to the capture probes 808). Second substrate 822 can include the first coating 806 (e.g., a conductive coating), thereby allowing electric field “-E” to be generated.

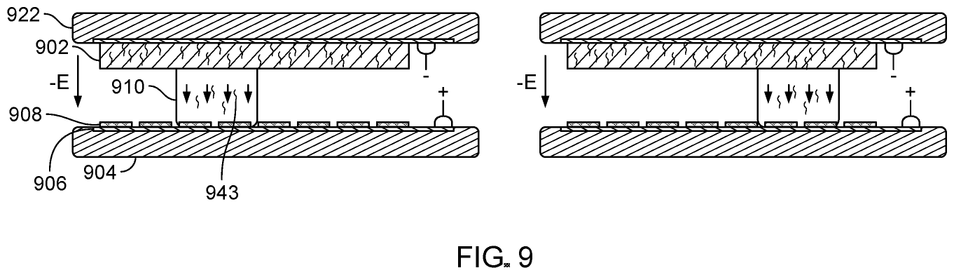

FIG. 9 shows an example schematic workflow for spatially transferring analytes 943, where the buffer 910 only contacts certain region of the biological sample 902 (e.g., a region of interest). In some embodiments, the buffer 910 only contacts a first region of the biological sample 902 so that only analytes 943 from the first region can be transferred to the capture probes 908.

In some embodiments, biological sample 902 includes analytes 943. In some embodiments, the analytes 943 are negatively charged. First substrate 904 can include an array of capture probes 908 that is fixed or attached to the first substrate 904 or attached to features (e.g., beads) on the substrate. In some embodiments, capture probes 908 can include any of the capture probes disclosed herein. In some embodiments, first substrate 902 does not include features and instead, capture probes 908 are directly attached to the substrate surface. In some embodiments, the capture probes 908 are positively charged.

Biological sample 902, a semi-porous material 99 and capture probes 908 (e.g., an array of capture probes) can be in contact with buffer 910. In some embodiments, buffer 910 contains a permeabilization reagent. Non-limiting examples of permeabilization reagents include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include, but are not limited to, dried permeabilization reagents, a permeabilization buffer, a buffer without a permeabilization reagent, a permeabilization gel, and a permeabilization solution. In some examples, biological samples (e.g., tissue samples) can be permeabilized first and then be subjected to electrophoresis.

In some embodiments, the analytes 943 can be proteins and/or nucleic acids. In some embodiments, the analytes 943 are negatively charged proteins and/or nucleic acids. In some embodiments, the capture probes 908 are proteins and/or nucleic acids. In some embodiments, the capture probes 908 are positively charged proteins and/or nucleic acids. In some embodiments, the analytes 943 are negatively charged transcripts. In some embodiments, the analytes 943 are poly(A) containing transcripts. In some embodiments, the capture probes 908 are attached to a feature in a feature array. In some embodiments, buffer 910 can be in contact with the biological sample 902, first substrate 904, second substrate 922, or any combination thereof.

In FIG. 9, the biological sample 902 can be subjected to electrophoresis. During electrophoresis, the biological sample 902 is subjected to an electric field that can be generated by sandwiching biological sample 902 between the first substrate 904 and a second substrate 922, connecting each substrate to a cathode and an anode, respectively, and running an electric current through the substrates. The application of the electric field “-E” causes the analytes 943 (e.g., negatively charged analytes) to migrate towards the substrate 904 and capture probes 908 in the direction of the arrows shown in FIG. 9. In some embodiments, the analytes 943 migrate towards capture probes 908 from the permeabilized biological sample (e.g., from an original location in a permeabilized cell to a final location in or close to the capture probes 908). Second substrate 922 can include the first coating 906 (e.g., a conductive coating), thereby allowing electric field “-E” to be generated.

g) Systems for Multiplex Analyte Capture

Also provided herein are system including (a) an array including a plurality of capture probes, where a capture probe of the plurality of capture probes includes (i) a spatial barcode and (ii) a capture domain; (b) one or more semi-porous materials disposed between a biological sample and the array; (c) an electric field, where the electric field promotes the migration of one or more analytes in the direction of the array, where the capture domain of the capture probe binds to a first analyte of the one or more analytes.

Various methods of generating an electric field are known in the art. Any suitable method of applying an electric field to the array (e.g., in some examples the first array, the second array, or both), the biological sample, and the one or more semi-porous materials.

In some embodiments, the first semi-porous material of the one or more semi-porous materials retains one or more second analytes. In some embodiments, the first semi-porous material includes a plurality of analyte-binding moieties, where an analyte-binding moiety of the plurality specifically binds to a second analyte of the one or more second analytes. In some embodiments, the analyte binding moiety is an antibody or antigen-binding fragment thereof.

In some embodiments, the second analyte is a protein.

In some embodiments, the system includes determining the location of the second analyte in the first semi-porous material. In some embodiments, determining the location of the second analyte in the first semi-porous material includes immunofluorescence staining. In some embodiments, determining the location of the second analyte in the first semi-porous material includes obtaining an image of the first semi-porous material and correlating the immunofluorescence staining in the image of the first semi-porous material with an image of the biological sample.

In some embodiments, the one or more semi-porous materials includes a second semi-porous material, where the second semi-porous material retains one or more third analytes. In some embodiments, the second semi-porous material includes a plurality of analyte-binding moieties for the one or more third analytes. In some embodiments, the analyte-binding moiety of the plurality of analyte binding moieties specifically binds to a third analyte of the one or more third analytes. In some embodiments, the third analyte is a nucleic acid. In some embodiments, the nucleic acid is miRNA. In some embodiments, the system includes a step of determining the location of the third analyte in the second semi-porous material. In some embodiments, determining the location of the third analyte in the second semi-porous material includes the use of in situ hybridization. In some embodiments, determining the location of the third analyte includes obtaining an image of the second semi-porous material and correlating the in situ hybridization in the image of the second semi-porous material with an image of the biological sample. In some embodiments, the system includes a step of crosslinking the one or more third analytes to the second semi-porous material.

In some embodiments, the system includes a step of cross-linking the one or more second analytes to the first semi-porous material. In some embodiments, removing the second semi-porous material after the second semi-porous material retains the one or more third analytes. In some embodiments, the system includes a step of removing the first semi-porous material after the first semi-porous material retains the one or more second analytes and disposing the second semi-porous material between the biological sample and the array.

In some embodiments, the electric field is applied after removal of the first semi-porous material and disposal of the second semi-porous material between the biological sample and the array. In some embodiments, the second semi-porous material retains one or more third analytes.

In some embodiments, the system includes a step of determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample.

In some embodiments, the capture probe further include one or more of: a cleavage domain, a functional domain, and a unique molecular identifier.

In some embodiments, the system includes imaging the biological sample. In some embodiments, the first analyte is mRNA. In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size. In some embodiments, the one or more semi-porous materials includes one or more fiducial markers that align the one or more semi-porous materials to the image of the biological sample. In some embodiments, the biological sample is a tissue section. In some embodiments, the biological sample is a formalin-fixed paraffin-embedded biological sample. In some embodiments, the system includes a step of fixing the biological sample. In some embodiments, the system includes a step of permeabilizing the biological sample. In some embodiments, the second analyte is protein. In some embodiments, an electric field is applied to a discrete area of the biological sample. In some embodiments, the electric field is applied to a discrete area of the array. In some embodiments, the biological sample, the semi-porous material, and the array, are in direct contact with a buffer.

Also provided herein are systems where one or more analytes can be captured on a second array. For example, analytes from a biological sample can be transferred through one or more semi-porous materials and captured on a first array. In some embodiments, the one or more semi-porous materials retain one or more analytes. In some embodiments, the one or more semi-porous materials include a plurality of analyte binding moieties (e.g., analyte binding moieties for nucleic acid). In some embodiments, the one or more semi-porous materials include a plurality of analyte capture agents. In some embodiments, the first array is replaced by a second array and analytes retained in the one or more semi-porous materials are captured on the second array. In some embodiments, a second electric field promotes the migration of the one or more analytes in the one or more semi-porous materials to the second array.

Thus, provided herein are systems including (a) a first array including a plurality of first capture probes, where a first capture probe of the plurality of first capture probes includes (i) a first spatial barcode and (ii) a first capture domain, (b) a semi-porous material, where the semi-porous material is disposed between the biological sample and the first array; (c) a first electric field, where the first electric field promotes the migration of the one or more analytes in the direction of the first array, where the first capture domain specifically binds to a first analyte of the one or more analytes, where the semi-porous material retains a second analyte of the one or more analytes; (d) a second array including a plurality of second capture probes, where a second capture probe of the plurality of second capture probes includes (i) a second spatial barcode and (ii) a second capture domain, (e) a second electric field, where the second electric field promotes the migration of the second analyte in the direction of the second array, where the second capture domain specifically binds to the second analyte; and (f) determining (i) all or a portion of the sequence of the second spatial barcode, or a complement thereof, and (ii) all or a portion of the sequence of the second analyte, or a complement thereof, and using the sequences of (i) and (ii) to determine the location of the second analyte in the biological sample.

In some embodiments, the capture probe includes one or more of: a cleavage domain, a functional domain, and a unique molecular identifier. In some embodiments, the first analyte is mRNA. In some embodiments, the system includes a step of obtaining an image of the biological sample. In some embodiments, the semi-porous material includes a hydrogel. In some embodiments, the semi-porous material includes a permeable membrane. In some embodiments, the semi-porous material has non-uniform pore sizes. In some embodiments, the semi-porous material has a substantially uniform pore size. In some embodiments, the one or more semi-porous materials includes one or more fiducial markers that align the one or more semi-porous materials to the image of the biological sample.

In some embodiments, the biological sample is a tissue section. In some embodiments, the biological sample is a formalin-fixed paraffin-embedded biological sample. In some embodiments, the system includes a step of fixing the biological sample. In some embodiments, the system includes a step of permeabilizing the biological sample. In some embodiments, the semi-porous material retains one or more analytes from the biological sample.

In some embodiments, the first electric field, the second electric field, or both, is applied to a discrete area of the biological sample. In some embodiments, where the first electric field is applied to a discrete area of the first array. In some embodiments, the second electric field is applied to a discrete area of the second array.

In some embodiments, the biological sample, the semi-porous material, and the first array are in direct contact with a buffer. In some embodiments, the biological sample, the semi-porous material and the second array are in direct contact with a buffer. In some embodiments, the semi-porous material separates one or more analytes from other molecules in the biological sample.

In some embodiments, the semi-porous material includes one or more analyte binding moieties. In some embodiments, the one or more analyte binding moieties bind a nucleic acid. In some embodiments, the nucleic acid is miRNA. In some embodiments, the analyte binding moiety is crosslinked to the semi-porous material.

In some embodiments, the semi-porous material includes one or more analyte capture agents. In some embodiments, the one or more analyte capture agents includes (i) an analyte binding moiety, (ii) analyte binding moiety barcode, and (iii) an analyte capture sequence. In some embodiments, the second analyte is protein. In some embodiments, the analyte capture agent is crosslinked to the semi-porous material. In some embodiments, the analyte binding moiety include an antibody or an antigen-binding fragment thereof. In some embodiments, the analyte binding moiety barcode identifies the analyte capture agent. In some embodiments, the analyte capture sequence binds to the second capture domain of the second capture probe.

In some embodiments, the system includes a step of (a) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample and (b)

determining (i) all or a portion of the sequence of the second spatial barcode, or a complement thereof, and (ii) all or a portion of the sequence of the analyte binding moiety barcode, or a complement thereof, and using the sequences of (i) and (ii) to determine the location of the second analyte in the biological sample.

In some embodiments, a system further comprises a substrate holder such as a cassette wherein any of the substrates can be placed. For example, a substrate holder (e.g., a cassette) can hold a biological sample on a substrate (e.g., a slide), a first array, or a second array. A substrate holder as previously described allows for a means by which to apply electrophoretic current to, a biological sample, a substrate, one or more semi-porous materials, and the like as described herein to separate the one or more analytes from a biological sample.

In some embodiments, a system further comprises a computer and a computer program by which to effect the application of an electrophoretic current in a system.

### EMBODIMENTS

Embodiment 1 is a method for determining a location of one or more analytes in a biological sample, the method comprising: (a) providing an array comprising a plurality of capture probes, wherein a capture probe of the plurality of capture probes comprises (i) a spatial barcode and (ii) a capture domain; (b) providing one or more semi-porous materials, wherein the one or more semi-porous materials are disposed between a biological sample and the array; (c) applying an electric field to the biological sample, the one or more semi-porous materials, and the array, wherein the electric field promotes the migration of the one or more analytes in the direction of the array, wherein the capture domain of the capture probe specifically binds to a first analyte of the one or more analytes; and (d) determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample.

Embodiment 2 is the method of embodiment 1, wherein the capture probe further comprises one or more of: a cleavage domain, a functional domain, and a unique molecular identifier.

Embodiment 3 is the method of embodiment 1 or 2, wherein the first analyte is mRNA.

Embodiment 4 is the method of any one of embodiments 1-3, wherein further comprising obtaining an image of the biological sample.

Embodiment 5 is the method of any one of embodiments 1-4, wherein a first semi-porous material of the one or more semi-porous materials retains one or more second analytes.

Embodiment 6 is the method of embodiment 5, wherein the first semi-porous material comprises a plurality of analyte-binding moieties, wherein an analyte-binding moiety of the plurality specifically binds to a second analyte of the one or more second analytes.

Embodiment 7 is the method of embodiment 6, wherein the analyte binding moiety is an antibody or an antigen-binding fragment thereof.

Embodiment 8 is the method of embodiment 6 or 7, wherein the second analyte is a protein.

Embodiment 9 is the method of any one of embodiments 5-8, further comprising determining the location of the second analyte in the first semi-porous material.

Embodiment 10 is the method of embodiment 9, wherein determining the location of the second analyte in the first semi-porous material comprises the use of immunofluorescence staining.

Embodiment 11 is the method of embodiment 10, wherein determining the location of the second analyte in the first semi-porous material comprises: obtaining an image of the first semi-porous material; and correlating the immunofluorescence staining in the image of the first semi-porous material with an image of the biological sample.

Embodiment 12 is the method of any one of embodiments 1, wherein the one or more semi-porous materials comprise a second semi-porous material, wherein the second semi-porous material retains one or more third analytes.

Embodiment 13 is the method of embodiment 12, wherein the second semi-porous material comprises a plurality of analyte-binding moieties for the one or more third analytes.

Embodiment 14 is the method of embodiment 13, wherein an analyte-binding moiety of the plurality of analyte binding moieties specifically binds to a third analyte of the one or more third analytes.

Embodiment 15 is the method of embodiment 14, wherein the third analyte is a nucleic acid.

Embodiment 16 is the method of embodiment 15, wherein the nucleic acid is miRNA.

Embodiment 17 is the method of any one of embodiments 12-16, further comprising determining the location of the third analyte in the second semi-porous material.

Embodiment 18 is the method of embodiment 17, wherein determining the location of the third analyte in the second semi-porous material comprises the use of in situ hybridization.

Embodiment 19 is the method of embodiment 18, wherein determining the location of the third analyte comprises: obtaining an image of the second semi-porous material; and correlating the in situ hybridization in the image of the second semi-porous material with an image of the biological sample.

Embodiment 20 is the method of embodiment 1, wherein the one or more semi-porous materials separate the one or more analytes from other molecules in the biological sample.

Embodiment 21 is the method of any one of embodiments 1-20, wherein the one or more semi-porous materials comprise a hydrogel.

Embodiment 22 is the method of any one of embodiments 1-20, wherein the one or more semi-porous materials comprise a permeable membrane.

Embodiment 23 is the method of any one of embodiments 1-22, wherein at least one of the one or more semi-porous materials has non-uniform pore sizes.

Embodiment 24 is the method of any one of embodiments 1-22, wherein at least one of the one or more semi-porous materials has a substantially uniform pore size.

Embodiment 25 is the method of any one of embodiments 1-24, wherein the electric field is applied to a discrete area of the biological sample.

Embodiment 26 is the method of any one of embodiments 1-24, wherein the electric field is applied to a discrete area of the array.

Embodiment 27 is the method of embodiment 4, wherein the one or more semi-porous materials comprise one or more fiducial markers that align the one or more semi-porous materials to the image of the biological sample.

Embodiment 28 is the method of any one of embodiments 1-27, wherein the biological sample, the one or more semi-porous materials, and the array are in direct contact with a buffer.

Embodiment 29 is the method of embodiment 5, further comprising crosslinking the one or more second analytes to the first semi-porous material.

Embodiment 30 is the method of embodiment 12, further comprising crosslinking the one or more third analytes to the second semi-porous material.

Embodiment 31 is the method of embodiment 5, further comprising removing the first semi-porous material after the first semi-porous material retains the one or more second analytes.

Embodiment 32 is the method of embodiment 12, further comprising removing the second semi-porous material after the second semi-porous material retains the one or more third analytes.

Embodiment 33 is the method of embodiment 12, wherein the method further comprises: removing the first semi-porous material after the first semi-porous material retains the one or more second analytes; and disposing the second semi-porous material between the biological sample and the array.

Embodiment 34 is the method of embodiment 33, wherein the electric field is applied after removal of the first semi-porous material and disposal of the second semi-porous material between the biological sample and the array.

Embodiment 35 is the method of embodiment 34, wherein the second semi-porous material retains one or more third analytes.

Embodiment 36 is the method of any one of embodiments 1-35, wherein the biological sample is a tissue section.

Embodiment 37 is the method of any one of embodiments 1-35, wherein the biological sample is a formalin-fixed paraffin-embedded biological sample.

Embodiment 38 is the method of any one of embodiments 1-35, further comprising a step of fixing the biological sample.

Embodiment 39 is the method of embodiments 1-35, further comprising a step of permeabilizing the biological sample.

Embodiment 40 is a method for determining a location of one or more analytes in a biological sample, the method comprising: (a) providing a first array comprising a plurality of first capture probes, wherein a first capture probe of the plurality of first capture probes comprises (i) a first spatial barcode and (ii) a first capture domain; (b) providing a semi-porous material, wherein the semi-porous material is disposed between the biological sample and the first array; (c) applying an electric field to the biological sample, the semi-porous material, and the first array, wherein the electric field promotes the migration of the one or more analytes in the direction of the first array, wherein the first capture domain specifically binds to a first analyte of the one or more analytes, wherein the semi-porous material retains a second analyte of the one or more analytes; (d) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample; (e) providing a second array comprising a plurality of second capture probes, wherein a second capture probe of the plurality of second capture probes comprises (i) a second spatial barcode and (ii) a second capture domain; (f) applying an electric field to the semi-porous material and the second array, wherein the electric field promotes the migration of the second analyte in the direction of the second array, wherein the second capture domain specifically binds to the second analyte; and (g) determining (i) all or a portion of the sequence of the second spatial barcode, or a complement thereof, and (ii) all or a portion of the sequence of the second analyte, or a complement thereof, and using the sequences of (i) and (ii) to determine the location of the second analyte in the biological sample.

Embodiment 41 is the method of embodiment 40, wherein the capture probe further comprises one or more of: a cleavage domain, a functional domain, and a unique molecular identifier.

Embodiment 42 is the method of embodiment 40 or 41, wherein the first analyte is mRNA.

Embodiment 43 is the method of any one of embodiments 40-42, wherein the method further comprises obtaining an image of the biological sample.

Embodiment 44 is the method of embodiment 43, wherein the semi-porous material comprises one or more analyte binding moieties.

Embodiment 45 is the method of embodiment 44, wherein the one or more analyte binding moieties specifically bind to a nucleic acid.

Embodiment 46 is the method of embodiment 45, wherein the nucleic acid is miRNA.

Embodiment 47 is the method of embodiment 40, wherein the semi-porous material retain one or more analytes in the biological sample.

Embodiment 48 is the method of any one of embodiments 40-47, wherein the semi-porous material comprises a hydrogel.

Embodiment 49 is the method of any one of embodiments 40-47, wherein the semi-porous material comprises a permeable membrane.

Embodiment 50 is the method of any one of embodiments 40-49, wherein the semi-porous material has non-uniform pore sizes.

Embodiment 51 is the method of any one of embodiments 40-49, wherein the semi-porous material has a substantially uniform pore size.

Embodiment 52 is the method of any one of embodiments 40-51, wherein an electric field is applied to a discrete area of the biological sample.

Embodiment 53 is the method of any one of embodiment 40-51, wherein the electric field is applied to a discrete area of the first array.

Embodiment 54 is the method of any one of embodiments 40-53, wherein the electric field is applied to a discrete area of the second array.

Embodiment 55 is the method of embodiment 43, wherein the semi-porous material comprises one or more fiducial markers that align the semi-porous material to the image of the biological sample.

Embodiment 56 is the method of any one of embodiments 40-55, wherein, in step (c), the biological sample, the semi-porous material, and the first array are in direct contact with a buffer.

Embodiment 57 is the method of any one of embodiments 40-56, wherein, in step (0, the semi-porous material and the second array are in direct contact with a buffer.

Embodiment 58 is the method of any one of embodiments 40-57, further comprising crosslinking the second analyte to the semi-porous material.

Embodiment 59 is the method of embodiments 40-58, wherein the biological sample is a tissue section.

Embodiment 60 is the method of embodiments 40-58, wherein the biological sample is formalin-fixed and paraffin-embedded.

Embodiment 61 is the method of embodiments 40-58, further comprising a step of fixing the biological sample.

Embodiment 62 is the method of embodiments 40-58, further comprising a step of permeabilizing the biological sample.

Embodiment 63 is a method for determining a location of one or more analytes in a biological sample, the method comprising: (a) providing a first array comprising a plurality of first capture probes, wherein a first capture probe of the plurality of first capture probes comprises (i) a first spatial barcode and (ii) a first capture domain; (b) providing a semi-porous material, wherein the semi-porous material is disposed between the biological sample and the first array, wherein the semi-porous material comprises an analyte capture agent comprising (i) an analyte binding moiety, (ii) analyte binding moiety barcode, and (iii) an analyte capture sequence; (c) applying an electric field to the biological sample, the semi-porous material, and the first array, wherein the electric field promotes the migration of the one or more analytes in the direction of the first array, wherein the first capture domain specifically binds to a first analyte of the one or more analytes, and wherein the analyte binding moiety specifically binds to a second analyte of the one or more analytes, and the semi-porous material retains the second analyte; (d) determining (i) all or a portion of the sequence of the first spatial barcode, or a complement thereof, and (ii) all or a portion of the sequence of the first analyte, or a complement thereof, and using the sequences of (i) and (ii) to determine the location of the first analyte in the biological sample; (e) providing a second array comprising a plurality of second capture probes, wherein a second capture probe of the plurality of second capture probes comprises (i) a second spatial barcode and (ii) a second capture domain; (f) applying an electric field to the semi-porous material and the second array, wherein the electric field promotes the migration of the second analyte in the direction of the second array, wherein the second capture domain specifically binds to the analyte capture sequence; and (g) determining (i) all or a portion of the sequence of the second spatial barcode, or a complement thereof, and (ii) all or a portion of the sequence of the analyte binding moiety barcode, or a complement thereof, and using the sequences of (i) and (ii) to determine the location of the second analyte in the biological sample.

Embodiment 64 is the method of embodiment 63, wherein one or both of the first capture probe and the second capture probe further comprises one or more of a cleavage domain, a functional domain, and a unique molecular identifier.

Embodiment 65 is the method of embodiment 63 or 64, wherein the first analyte is mRNA.

Embodiment 66 is the method of any one of embodiments 63-65, wherein the method further comprises obtaining an image of the biological sample.

Embodiment 67 is the method of any one of embodiments 63-66, wherein the analyte capture agent is crosslinked to the semi-porous material.

Embodiment 68 is the method of any one of embodiments 63-66, wherein the second analyte is a protein.

Embodiment 69 is the method of any one of embodiments 63-67, wherein the analyte binding moiety comprises an antibody or an antigen-binding fragment thereof.

Embodiment 70 is the method of any one of embodiments 63-69, wherein the semi-porous material separates one or more analytes from other molecules in the biological sample.

Embodiment 71 is the method of any one of embodiments 63-70, wherein the semi-porous material comprises a hydrogel.

Embodiment 72 is the method of any one of embodiments 63-70, wherein the semi-porous material comprises a permeable membrane.

Embodiment 73 is the method of any one of embodiments 63-72, wherein the semi-porous material has non-uniform pore sizes.

Embodiment 74 is the method of any one of embodiments 63-72, wherein the semi-porous material has a substantially uniform pore size.

Embodiment 75 is the method of any one of embodiments 63-74, wherein the electric field is applied to a discrete area of the biological sample.

Embodiment 76 is the method of any one of embodiments 63-75, wherein the electric field is applied to a discrete area of the first array.

Embodiment 77 is the method of any one of embodiments 63-76, wherein the electric field is applied to a discrete area of the second array.

Embodiment 78 is the method of embodiment 66, wherein the semi-porous material comprises one or more fiducial markers that align the semi-porous material to the image of the biological sample.

Embodiment 79 is the method of any one of embodiments 63-74, wherein, during step (c), the biological sample, the semi-porous material, and the first array, are in direct contact with a buffer.

Embodiment 80 is the method of any one of embodiments 63-75, wherein, during step (f), the semi-porous material and the second array are in direct contact with a buffer.

Embodiment 81 is the method of any one of embodiments 63-76, further comprising crosslinking the second analyte to one or both of the semi-porous material and the analyte capture agent.

Embodiment 82 is the method of embodiments 63-81, wherein the biological sample is a tissue section.

Embodiment 83 is the method of embodiments 63-81, wherein the biological sample is formalin-fixed and paraffin-embedded.

Embodiment 84 is the method of embodiments 63-81, further comprising a step of fixing the biological sample. Embodiment 85 is the method of embodiments 63-81, further comprising a step of permeabilizing the biological sample.

1-156. (canceled)

157. A method for determining a location of one or more analytes in a biological sample, the method comprising:
(a) providing an array comprising a plurality of capture probes, wherein a capture probe of the plurality of capture probes comprises: (i) a spatial barcode and (ii) a capture domain;
(b) providing one or more semi-porous materials, wherein the one or more semi-porous materials are disposed between a biological sample and the array;
(c) applying an electric field to the biological sample, the one or more semi-porous materials, and the array, wherein the electric field promotes the migration of the one or more analytes in the direction of the array, wherein the capture domain of the capture probe specifically binds to a first analyte of the one or more analytes, wherein the first analyte is a nucleic acid; and
(d) determining (i) the sequence of the spatial barcode, or a complement thereof, and (ii) all or a portion of the sequence of the nucleic acid, or a complement thereof, and using the sequences of (i) and (ii) to determine the location of the first analyte in the biological sample.

158. The method of claim 157, wherein the capture probe further comprises one or more of: a cleavage domain, a functional domain, and a unique molecular identifier.

159. The method of claim 157, further comprising imaging the biological sample.

160. The method of claim 157, wherein a first semi-porous material of the one or more semi-porous materials retains one or more second analytes, and wherein the first analyte is a mRNA and a second analyte is a protein.

161. The method of claim 160, wherein the first semi-porous material comprises a plurality of protein-binding moieties that specifically bind to one or more proteins.

162. The method of claim 161, further comprising determining the location of the one or more proteins in the first semi-porous material, wherein determining the location of the one or more proteins in the first semi-porous material comprises the use of immunofluorescence staining and imaging the first semi-porous material; and correlating the immunofluorescence staining in the image of the first semi-porous material with an image of the biological sample.

163. The method of claim 157, wherein the one or more semi-porous materials comprises a second semi-porous material, wherein the second semi-porous material retains one or more third analytes, wherein the second semi-porous material comprises a plurality of analyte-binding moieties for the one or more third analytes.

164. The method of claim 163, wherein an analyte-binding moiety of the plurality of analyte binding moieties specifically binds to a third analyte of the one or more third analytes, wherein the third analyte is a nucleic acid.

165. The method of claim 164, further comprising determining the location of the third analyte in the second semi-porous material, wherein determining the location of the third analyte in the second semi-porous material comprises in situ hybridization.

166. The method of claim 165, wherein determining the location of the third analyte comprises:
imaging the second semi-porous material; and correlating the in situ hybridization in the image of the second semi-porous material with an image of the biological sample.

167. The method of claim 157, wherein the one or more semi-porous materials comprise a hydrogel or a permeable membrane.

168. The method of claim 157, wherein at least one of the one or more semi-porous materials has non-uniform pore sizes.

169. The method of claim 157, wherein at least one of the one or more semi-porous materials has a substantially uniform pore size.

170. The method of claim 157, wherein the electric field is applied to a discrete area of the biological sample or is applied to a discrete area of the array.

171. The method of claim 157, wherein the one or more semi-porous materials comprise one or more fiducial markers that align the one or more semi-porous materials to the image of the biological sample.

172. The method of claim 157, wherein the biological sample, the one or more semi-porous materials, and/or the array are in direct contact with a buffer and wherein the biological sample is a tissue section or a fixed tissue section.

173. The method of claim 163, further comprising crosslinking the one or more second analytes to the first semi-porous material, and optionally, crosslinking the one or more third analytes to the second semi-porous material.

174. The method of claim 160, further comprising removing the first semi-porous material after the first semi-porous material retains the one or more proteins, and optionally, removing the second semi-porous material after the second semi-porous material retains the one or more third analytes.

175. The method of claim 160, wherein the method further comprises:
removing the first semi-porous material after the first semi-porous material retains the one or more proteins;
disposing the second semi-porous material between the biological sample and the array; and
applying the electric field after removal of the first semi-porous material and disposal of the second semi-porous material between the biological sample and the array.

176. The method of claim 157, further comprising a step of fixing and/or permeabilizing the biological sample.
